(19) United States
(12) Patent Application Publication
(10) Pub. No.: US 2005/0020654 A1
(43) Pub. Date: Jan. 27, 2005

(54) Novel PPAR agonists, pharmaceutical compositions and uses thereof

(76) Inventors: Harrihar A. Pershadsingh, Bakersfield, CA (US); Mitchell A. Avery, Oxford, MS (US)

Correspondence Address: COOLEY GODWARD, LLP, 3000 EL CAMINO REAL, 5 PALO ALTO SQUARE, PALO ALTO, CA, 94306, US

(21) Appl. No.: 10/801,437

(22) Filed: Mar. 15, 2004

Related U.S. Application Data
(60) Provisional application No. 60/455,375, filed on Mar. 15, 2003.

(51) Int. Cl. A61K 31/4184; C 07D 4 3/02

(52) U.S. Cl. 514/394; 548/304.7; 514/338; 546/273.4

(57) ABSTRACT

The present invention provides novel compounds and pharmaceutical compositions thereof, which at least partially activate PPARγ and may further inhibit the activity of the AT1 receptor. The novel compounds include certain substituted benzimidazole compounds of Formulae I and II, infra. The invention also provides methods of treating inflammatory and metabolic disorders and methods for screening compounds for the capability to treat or prevent an inflammatory or metabolic disorder.

This application claims benefit under 35 U.S.C. § 119(e) of U.S. Provisional Application Ser. No. 60/455,375, filed on Mar. 15, 2003, which is herein incorporated by reference in its entirety for all purposes.

### 1. TECHNICAL FIELD

This invention relates to the field of prevention and treatment of inflammatory and metabolic disorders, in particular, obesity and weight gain, and insulin resistance syndromes. More specifically, this invention relates to compounds that partially activate the PPARγ isoform and which may also inhibit the angiotensin II type 1 receptor (AT1).

### 2. BACKGROUND

Peroxisome proliferator-activated receptors (PPARs) are members of the nuclear receptor superfamily of ligand-activated transcription factors. Three subtypes of PPARs have been isolated from mouse and human sources, i.e., PPARα, PPARγ, and PPARδ (Willson et al., Annu Rev Biochem. 2001, 70:341-367). The PPARs are important regulators of intermediary (carbohydrate, lipid and protein) metabolism, energy metabolism, cell growth, cell differentiation, cell maturation, phenotype transition, apoptosis, neovascularization, angiogenesis, inflammation, immune regulation and the immune response. Compounds that activate PPARs are useful for the treatment and/or prevention of a variety of clinical disorders, including but not limited to, metabolic disorders, cardiovascular disorders, neurodegenerative disorders, autoimmune disorders, immunoregulatory disorders, metabolic syndrome, obesity, pre-diabetes, type 2 diabetes and other insulin resistant syndromes, hypertension, atherosclerosis, dyslipidemia, inflammatory skin diseases (e.g., psoriasis) inflammatory bowel disease and inflammatory neurodegenerative diseases (e.g., multiple sclerosis and Alzheimer's disease). (Pershadsingh, Expert Opin Investig Drugs. 1999, 8:1859-1872; Debril et al., J Mol Med. 2001, 79:30-47; Delerive et al., J Endocrinol. 2001;169:453-9; Clark, J Leukoc Biol. 2002, 71:388-400; Ellis et al., Arch Dermatol. 2000, 136:609-16; Lewis et al. Am J Gastroenterol. 2001, 6:3323-8; Landreth et al., Neurobiol Aging. 2001, 22:937-44; Feinstein et al., Ann Neurol. 2002, 51:694-702). Metabolic syndrome includes the metabolic syndrome as defined by either the World Health Organization (WHO) or the National Cholesterol Education Program (NCEP) (Zimmet et al., Nature 2001, 414:782-7; Alberti et al., Diabet Med. 1998, 15:539-53).

Compounds which activate PPARs include, but are not limited to, thiazolidinediones (e.g., rosiglitazone, pioglitazone, troglitazone, MK 767 (KRP-297), MCC-555, netoglitazone, balaglitazone, rivoglitazone, CLX-0921, R-483, NIP-221, NIP-223, DRF-2189), and the like that primarily activate PPARγ or PPARγ and PPARα, non-thiazolidinediones that can activate any combination of PPARγ, PPARα and PPARδ (e.g., JTT-501, LSN862, DRF 4832, LM 4156, LY 510929, LY 519818, TY 51501, X 334, tesaglitazar, farglitazar, GW-7282, TAK-559, T-131, RG-12525, LY-510929, LY-519818, BMS-298585, DRF-2725, GW-1536, GI-262570, TZD18 (Merck), DRF-2655, and the like), certain tyrosine-based derivatives (e.g., GW1929, GW7845), phenylacetic acid-based derivatives, phenoxazine phenyl propanoic acid derivatives (e.g., DRF 2725, DRF 2189), cinammic and dihydrocinammic acid-based derivatives (e.g., tesaglitazar (AZ 242)), and 3-phenyl-7-propylbenzisoxazoles (Adams et al., Bioorg Med Chem Lett. 2003, 13:931-5), that can activate PPARγ in combination with PPARα or PPARδ or both PPARα and PPARδ (also see Table 1, in: Miller A R, Etgen G J. Expert Opin Investig Drugs. 2003, 12:1489-500). Although some compounds primarily activate PPARα alone or PPARδ alone, more commonly such compounds also activate, to least some degree, PPARγ.

Compounds which antagonize the angiotensin II type 1 receptor and also activate PPARγ include, but are not limited to 2-methyl-2-[4-(2-[5-methyl-2-aryloxazol-4-yl]ethoxy)phenoxy]propionic acid PPARα/γ agonist derivatives (Brooks et al., J Med Chem 2001, 44:2061-4) N-(2-Benzoylphenyl)-L-tyrosine PPARγ agonists (Henke et al., J Med Chem 1998, 41:5020-36; dihydrocinnamate PPARα/γ agonist derivatives (Cronet et al., Structure 2001, 9:699-706. Another angiotensin II type 1 receptor blocker (ARB) which can be optionally derivatized to also fully or partially activate PPARγ are heterocyclic benzimidazoles (U.S. Pat. No. 6,100,252).

Compounds, such as those above, which are full agonists of PPARγ may be used to treat and/or prevent type 2 diabetes and a variety of other disorders. However, those compounds that fully activate PPARγ are associated with numerous adverse effects (e.g., weightgain, fluid retention, peripheral edema, pulmonary edema and congestive heart failure) which limits the clinical utility of these ligands.

In contrast to full PPARγ agonists, PPARγ partial agonists or synthetic PPARγ modulators (SPPARMs) may possess substantial efficacy with reduced side-effect profiles, including diminished potential for weight gain, fluid retention and edema.

Accordingly, what is needed are novel compounds which are PPARγ partial agonists. Ideally, these compounds may be used to treat and/or prevent type 2 diabetes and a variety of other disorders without significant side effects. The PPARγ partial agonists may also inhibit the angiotensin II type 1 receptor (AT1).

### 3. SUMMARY

The present invention satisfies these and other needs by providing novel compounds which at least partially activate PPARγ and may further inhibit the activity of the AT1 receptor. The novel compounds include compounds of Formulae I and II, infra.

In a second aspect, the present invention provides pharmaceutical compositions of compounds of Formulae I and II, infra. The pharmaceutical compositions generally comprise one or more compounds of Formulae I and/or II and a pharmaceutically acceptable vehicle. In a preferred embodiment, the pharmaceutical compositions are for the treatment or prevention of a family of related metabolic disorders in a mammal, particularly a disorder selected from the group consisting of type 2 diabetes and the metabolic syndrome.

In a third aspect, the present invention provides methods for treating or prophylactically preventing an inflammatory or metabolic disorder in a mammal comprising administering to the mammal in need thereof, a therapeutically effective amount of a compound sufficient to at least partially activate a peroxisome proliferator-activated receptor (PPAR), in particular PPARγ. These methods can be used for treating or preventing a variety of inflammatory and proliferative diseases (see Tables I-X, infra), including but not limited to, type 2 diabetes and metabolic syndrome. The methods generally involve administering to a patient in need of such treatment or prevention a therapeutically effective amount of a compound of Formulae I and/or II, infra.

In a fourth aspect, the current invention provides methods of screening a compound for capability to treat or prevent an inflammatory or metabolic disorder in a mammal, the method comprising: (a) identifying a compound as at least partially activating a peroxisome proliferator-activated receptor (PPAR), particularly PPARγ; (b) identifying the compound as at least partially inhibiting an activity of angiotensin II type 1 receptors; and (c) selecting the compound as capable of treating or preventing an inflammatory or metabolic disorder. These methods may further comprise selecting a compound that does not cause, promote, or aggravate at least one of fluid retention, peripheral edema, pulmonary edema, and congestive heart failure in the mammal.

### 4. DETAILED DESCRIPTION

### 4.1 Definitions

“Compounds” refers to any compounds encompassed by generic formulae disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, that brackets indicate the point of attachment of the partial structure to the rest of the molecule.

The term “alkyl” refers to a monovalent, saturated aliphatic hydrocarbon radical having the indicated number of carbon atoms and that is optionally substituted. For example, a “C 1-6 alkyl” or an “alkyl of 1-6 carbons” or “Alk 1-6 would refer to any alkyl group containing one to six carbons in the structure. “C 1-20 alkyl” refer to any alkyl group having one to twenty carbons. Alkyl may be a straight chain (i.e. linear) or a branched chain. Lower alkyl refers to an alkyl of 1-6 carbons. Representative examples lower alkyl radicals include methyl, ethyl, n-propyl, n-butyl, n-pentyl, n-hexyl, isopropyl, isobutyl, isopentyl, amyl, sec-butyl, tert-butyl, tert-pentyl and the like. Higher alkyl refers to alkyls of seven carbons and above. These include n-heptyl, n-octyl, n-nonyl, n-decyl, n-dodecyl, n-tetradecyl, n-hexadecyl, n-octadecyl, n-eicosyl, and the like, along with branched variations thereof. The radical may be optionally substituted with substituents at positions that do not significantly interfere with the preparation of compounds falling within the scope of this invention and that do not significantly reduce the efficacy of the compounds. The alkyl is optionally substituted with one to five substituents independently selected from the group consisting of halo, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

“Alkenyl” by itself or as part of another substituent refers to an unsaturated branched, straight-chain, or cyclic aliphatic hydrocarbon radical having at least one carbon-carbon double bond derived by the removal of one hydrogen atom from a single carbon atom of a parent alkene having the indicated number of carbon atoms, e.g. 1-20, preferably 1-6. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. This radical is optionally substituted similarly to alkyl.

“Alkynyl” by itself or as part of another substituent refers to an unsaturated branched, straight-chain, or cyclic aliphatic hydrocarbon radical having at least one carbon-carbon triple bond derived by the removal of one hydrogen atom from a single carbon atom of a parent alkyne having the indicated number of carbon atoms, e.g. 1-20, preferably 1-6. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. This radical is optionally substituted similarly to alkyl.

“Acyl” by itself or as part of another substituent refers to a radical —C(O)R³⁰, where R³⁰ is hydrogen, alkyl, akenyl, alkynl, cycloalkyl, cycloalkenyl, cycloalkynyl, aryl, or arylalkyl, as defined herein. Representative examples include, but are not limited to formyl, acetyl, cyclohexylcarbonyl, cyclohexylmethylcarbonyl, benzoyl, benzylcarbonyl and the like.

The term “alkoxy” refers to a monovalent radical of the formula RO—, where R is an alkyl as defined herein. Lower alkoxy refers to an alkoxy of 1-6 carbon atoms, with higher alkoxy is an alkoxy of seven or more carbon atoms. Representative lower alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Higher alkoxy radicals include those corresponding to the higher alkyl radicals set forth herein. The radical may be optionally substituted with substituents at positions that do not significantly interfere with the preparation of compounds falling within the scope of this invention and that do not significantly reduce the efficacy of the compounds. The alkyl is optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

“Alkoxycarbonyl” by itself or as part of another substituent refers to a radical —C(O)OR³² where R³² represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

The term “alkylcarboxyloxy” is a monovalent radical having the formula —OC(O)Alk, where Alk is alkyl, preferably lower alkyl.

The term “alkylcarbonylamino” is a monovalent radical having the formula —NHC(O)Alk, where Alk is alkyl, preferably lower alkyl.

“Alkylsulfinyl” by itself or as part of another substituent refers to the radical R³³SO— where R³³ is hydrogen, alkyl, cycloalkyl or aryl as defined herein.

“Alkylsulfonyl” by itself or as part of another substituent refers to the radical R³³SO₂— where R³³ is hydrogen, alkyl, cycloalkyl or aryl as defined herein.

“Alkylsulfonylamino” by itself or as part of another substituent refers to the radical —NR³³SO₂R³⁴ where R³³ and R³⁴ independently are hydrogen, alkyl, cycloalkyl or aryl as defined herein.

“Alkylthio” by itself or as part of another substituent refers to the radical —SR³³ where R³³ is hydrogen, alkyl, cycloalkyl or aryl as defined herein.

“Amido” by itself or as part of another substituent refers to the radical —NR³³C(O)OH where R³³ is hydrogen, alkyl, cycloalkyl or aryl as defined herein.

“Amino” by itself or as part of another substituent refers to the radical —NR³⁴R³⁵ where R³⁴ and R³⁵ independently are hydrogen, alkyl, cycloalkyl or aryl as defined herein.

“Angiogenesis” refers to a process by which normally quiescent endothelium responds to physiological or pathological stimuli (such as proliferating endometrium, injury, tumor growth, or diabetic retinopathy) resulting in pathological proliferation of blood vessels (neovascularization). Pathological angiogenesis (neovascularization) results in inappropriate vascular proliferation as in tumor neovascularization, lymphangiogenesis, tumor metastasis, etc.

“Angiotensin II-dependent disease” refers to a disease in which: 1) administration of a AT1 receptor antagonist slows, ameliorates, stops or reverses the pathological process, and/or 2) said disease is associated with impaired signal transduction within the rennin-angiotensin-aldosterone-system (RAAS) system, and/or 3) said disease is facilitated or exacerbated by activation of the AT1 receptor by angiotensin II, the initiating step being the by binding of angiotensin II the AT1 receptor.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, phenyl, 1-naphthyl, 2-naphthyl, and the like. A “1-naphthyl” or “2-naphthyl” is a radical formed by removal of a hydrogen from the 1- or 2-position of a naphthalene structure, respectively. It is optionally substituted with from one to four substituents independently selected from the group consisting of halo, lower alkyl, lower alkoxy, hydroxy, cyano, nitro, amino, halogenated lower alkyl, formyl, halogenated lower alkoxy, hydroxycarbonyl, lower alkoxycarbonyl, lower alkylcarbonyloxy, and lower alkylcarbonylamino. A “phenyl” is a radical formed by removal of a hydrogen from a benzene ring. The phenyl is optionally substituted with from one to five substituents independently selected from the group consisting of halo, lower alkyl, lower alkoxy, hydroxy, cyano, nitro, amino, halogenated lower alkyl, halogenated lower alkoxy, carbonyl, hydroxycarbonyl, lower alkylcarbonyloxy, benzyloxy, optionally substituted piperidino, lower alkoxycarbonyl, and lower alkylcarbonylamino.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenyleth-1-yl, naphthylmethyl, 2-naphthyleth-1-yl, naphthobenzyl, 2-naphthophenyleth-1-yl and the like. Preferably, an arylalkyl group is (C₆-C₃₀) arylalkyl, e.g., the alkyl moiety of the arylalkyl group is (C₁-C₁₀) and the aryl moiety is (C₆-C₂₀). More preferably, an arylkyl group is (C₆-C₂₀) arylalkyl, e.g., the alkyl moiety of the arylalkyl group is (C₁-C₈) and the aryl moiety is (C₆-C₁₂).

“Body mass index” (BMI) refers to the weight in kilograms of a patient divided by the square of the height in meters, such that BMI has units of kg/m².

“Carbamoyl” by itself or as part of another substituent refers to the radical —C(O)NR⁶⁴R⁶⁵ where R⁶⁴ and R⁶⁵ are independently H—, alkyl, cycloalkyl, aryl, alkenyl, or alkynyl, or R⁶⁴ and R⁶⁵ together with the nitrogen atom form a cyclic amino.

“Carbamoyloxy” by itself or as part of another substituent refers to the radical —OC(O)NR⁴⁰R⁴¹ where each of R⁴⁰ and R⁴¹ is independently hydrogen, lower alkyl, hydroxy lower alkyl, alkoxy lower alkyl, amino lower alkyl, lower cycloalkyl, phenyl (substituted or unsubstituted), or benzyl (substituted or unsubstituted), or where R⁴⁰ and R⁴¹ together form a cyclic amino with the nitrogen atom. Examples include aminocarbonyloxy, methylaminocarbonyloxy, dimethyl aminocarbonyloxy, [4-(1-piperidino)-1-piperidino]carbonyloxy, 1-morpholinocarbonyloxy, 1-pyrrolidinyl, 1-piperazinecarbonyloxy, and the like.

Congestive heart failure” (HF) refers to heart failure of any etiology, including but not limited to, heart failure with diastolic dysfunction, heart failure with systolic dysfunction, heart failure associated with cardiac hypertrophy, and heart failure that develops as a result of infectious myocarditis, inflammatory myocarditis, chemical myocarditis, cardiomyopathy of any etiology, hypertrophic cardiomyopathy, congenital cardiomyopathy, and cardiomyopathy associated with ischemic heart disease or myocardial infarction.

The term “cycloalkyl” refers to a monovalent, alicyclic, saturated hydrocarbon radical having three or more carbons forming the ring. While known cycloalkyl compounds may have up to 30 or more carbon atoms, generally there will be three to seven carbons in the ring. The latter include, for example, cyclopropyl, cyclobutyl, cyclopentyl, cyclohexyl, and cycloheptyl. The radical may be optionally substituted with substituents at positions that do not significantly interfere with the preparation of compounds falling within the scope of this invention and that do not significantly reduce the efficacy of the compounds. The cycloalkyl is optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

A “cyclic amino” is a monovalent radical of saturated 5-, 6-, or 7-membered cyclic amine ring having no more than one additional hetero atom such as nitrogen, oxygen, or sulfur. Representative examples include, e.g., 1-pyrrolidino, 1-piperidino, morpholino, piperazino, and the like. These may be substituted or unsubstituted. If substituted, generally they will have no more than 2 substituents chosen from lower alkyl, lower cycloalkyl, hydroxy lower alkyl, phenyl (substituted or unsubstituted), benyzl (substituted or unsubstituted), aminocarbonylmethyl, lower alkylaminocarbonylmethyl, amino, mono-or di-lower alkylamino, or cyclic amino.

“Degenerative disease” refers to a disease associated with deterioration or destruction normal tissue, resulting from immune dysregulation resulting in the upregulation of one or more inflammatory nuclear transcription factors, inflammatory cytokines and other inflammatory molecules such as proteases (e.g., MMP-9) and iNOS, leading to pathological degeneration of the respective cell or tissue or organ which is the therapeutic target.

“Euglycemia” refers to a condition in which a patient has a fasting blood glucose concentration within the normal range, greater than 70 mg/dl (3.89 mmol/L) and less than 110 mg/dl (6.11 mmol/L).

A “fused 2-, 3-, or 4-ring heterocyclic radical” is polynuclear in that the adjacent rings share a pair of atoms, generally carbon atoms. At least one of the rings will be heterocyclic in that it will have a noncarbon atom such as nitrogen, oxygen, or sulfur. The ring system may contain from 9 to 18 atoms. A 2-ring heterocyclic system will generally have 9 or 10 atoms included in the ring. Examples of such a 2-ring system include benzimidazole, quinoline, isoquinoline, purine, indolizine, 4H-quinolizine, 3H-pyrrolizine, coumaran, coumarin, isocoumarin, 4-methylcoumarin, 3-chloro-H-methylcoumarin, chromone, benzofuran, benzothiophene, benzothiazole, indole, and the like. A 3-ring system will generally have 12 to 14 atoms included in the ring. Examples of such a 3-ring system include carbazole, acridine, and the like. A 4-ring fused system will generally have 16 to 18 atoms included in the chain. Examples of such a 4-ring system include isothebaine and the like. The ring is bonded through a carbon in the ring system. The radical may be optionally substituted with substituents at positions that do not significantly interfere with the preparation of compounds falling within the scope of this invention and that do not significantly reduce the efficacy of the compounds. The radical is optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

A “halo” substitutent is a monovalent halogen radical chosen from chloro, bromo, iodo, and fluoro. A “halogenated” compound is one substituted with one or more halo substituent.

“Heart failure” includes congestive heart failure, heart failure with diastolic dysfunction, heart failure with systolic dysfunction, heart failure associated with cardiac hypertrophy, and heart failure that develops as a result of chemically induced cardiomyopathy, congenital cardiomyopathy, and cardiomyopathy associated with ischemic heart disease or myocardial infarction.

A “5-membered heterocyclic ring” is a monovalent radical of a 5-member closed ring containing carbon and at least one other element, generally nitrogen, oxygen, or sulfur and may be fully saturated, partially saturated, or unsaturated (i.e. aromatic in nature). Generally the heterocycle will contain no more than two hetero atoms. Representative examples of unsaturated 5-membered heterocycles with only one hetero atom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolidinyl, 2- or 3-tetrahydrofuranyl, and 2- or 3-tetrahydrothiophenyl. Representative unsaturated 5-membered heterocyclic radicals having two hetero atoms include imidazolyl, oxazolyl, thiazolyl, pyrazolyl, tetrazolyl and the like. The corresponding fully saturated and partially saturated radicals are also included. The heterocyclic radical is bonded through an available carbon atom in the heteocyclic ring. The radical may be optionally substituted with substituents at positions that do not significantly interfere with the preparation of compounds falling within the scope of this invention and that do not significantly reduce the efficacy of the compounds. The ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

A “6-membered heterocyclic ring” is a monovalent radical of a 6-member closed ring containing carbon and at least one other element, generally nitrogen, oxygen, or sulfur and may be fully saturated, partially saturated, or unsaturated (i.e. aromatic in nature). Generally the heterocycle will contain no more than two hetero atoms. Representative examples of unsaturated 6-membered heterocycles with only one hetero atom include 2-, 3-, or 4-pyridinyl, 2H-pyranyl, and 4H-pryanyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two hetero atoms include 3- or 4-pyridazinyl, 2-, 4-, or 5-pyrimidinyl, 2-pyrazinyl, and the like. The corresponding fully saturated and partially saturated radicals are also included, e.g. 2-piperazine. The heterocyclic radical is bonded through an available carbon atom in the heterocyclic ring. The radical may be optionally substituted with substituents at positions that do not significantly interfere with the preparation of compounds falling within the scope of this invention and that do not significantly reduce the efficacy of the compounds. The ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

The term “hydroxycarbonyl” is a monovolent radical having the formula —C(O)OH.

“Inflammatory disease” refers to a disease associated dysfunction of the immune system, exemplified as, but not limited to: 1) increased production of inflammatory cytokines (interleukin (IL)-1beta, IL-2, IL-6, IL-8, IL-12, tumor necrosis factor-α, interferon-γ, monocyte chemoattractant protein-1), 2) increased conversion of Th2 lymphocytes to the Th1 phenotype or increased Th1/Th2 ratio, 3) inappropriate function of NK (killer) T lymphocytes resulting in auto-antibodies and lack of “self” recognition resulting in an autoimmune disease, 4) increased expression or activation of inflammatory nuclear transcription factors (NFAT, NF-κB, AP-1, JNK/STAT), 5) increased expression of iNOS.

“Insulin resistance” refers to a condition in which circulating insulin levels in excess of the normal response to a glucose load are required to maintain the euglycemic state (Ford et al., JAMA. 2002, 287:356-9). Insulin resistance and the response of a patient with insulin resistance to therapy, may be quantified by assessing the homeostasis model assessment to insulin resistance (HOMA-IR) score, a reliable indicator of insulin resistance (Katsuki et al., Diabetes Care 2001, 24:362-5). An estimate of insulin resistance by the homeostasis assessment model (HOMA)-IR score may be calculated by a formula disclosed in Galvin et al., Diabet Med 1992, 9:921-8 where HOMA-IR=[fasting serum insulin (μU/mL)]×[fasting plasma glucose (mmol/L)/22.5].

“Hyperinsulinemia” is defined as the condition in which a subject with insulin resistance, with or without euglycemia, in which the fasting or postprandial serum or plasma insulin concentration is elevated above that of normal, lean individuals without insulin resistance, having a waist-to-hip ration<1.0 (for men) or <0.8 (for women).

“Impaired glucose tolerance” (IGT), refers to a condition in which a patient has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L).

“Metabolic syndrome” is a syndrome complex consisting of multiple clinical conditions and risk factors that stratify together. The cardinal feature of the metabolic syndrome is insulin resistance (Laaksonen et al., Am J Epidemiol 2002, 156:1070-7). In one aspect, according to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference>40 inches or 102 cm in men, and >35 inches or 94 cm in women
2. Triglycerides:≧150 mg/dL
3. HDL-cholesterol<40 mg/dL in men
4. Blood pressure≧130/85 mm Hg (SBP≧130 or DBP≧85)
5. Fasting blood glucose≧110 mg/dL

In other aspects, the metabolic syndrome is described by accepted synonyms, which includes, but is not limited to, syndrome X, insulin resistance syndrome, insulin-resistant hypertension, the metabolic hypertensive syndrome, dysmetabolic syndrome. Components of the metabolic syndrome include, but is not limited to, glucose intolerance, impaired glucose tolerance, impaired fasting serum glucose, impaired fasting blood glucose, hyperinsulinemia, pre-diabetes, obesity, visceral obesity, hypertriglyceridemia, elevated serum concentrations of free fatty acids, elevated serum concentrations of C-reactive protein, elevated serum concentrations of lipoprotein(a), elevated serum concentrations of homocysteine, elevated serum concentrations of small, dense low-density lipoprotein (LDL)-cholesterol, elevated serum concentrations of lipoprotein-associated phospholipase (A2), reduced serum concentrations of high density lipoprotein (HDL)-cholesterol, reduced serum concentrations of HDL(2b)-cholesterol, reduced serum concentrations of adiponectin, and albuminuria (see: Pershadsingh HA. Peroxisome proliferator-activated receptor-gamma: therapeutic target for diseases beyond diabetes: quo vadis? Expert Opin Investig Drugs. (2004) 13:215-28, and references cited therein).

“Negatively charged group” refers to a moiety having a negative charge, either localized or delocalized. The term “negatively charged also encompasses thosemoieties that can be metabolized in vivo to a moiety having a negative charge. Exemplary negatively charged groups include 2-tetrazolyl, carboxyl, alkoxycarbonyl, carbamoyl, sulfonamido, or alkylsulfonamido. A lower alkoxycarbonyl, for example, can be metabolized in vivo to carboxyl, which is a negatively charged group.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Proliferative disease” refers to a disease associated with: 1) pathological proliferation of normally quiescent cells, 2) pathological migration of cells from their normal location (e.g. metastasis of neoplastic cells), 3) pathological expression of proteolytic enzymes such as the matrix metalloproteinases (collagenases, gelatinases, elastases), 4) pathological angiogenesis as in proliferative retinopathy and tumor metastasis.

“Obesity” refers to the condition where a patient has a BMI equal to or greater than 30 kg/m².

“Overweight” refers to a patient with a BMI greater than or 25 kg/m² and less than 30 kg/m².

“Patient” refers to a mammal, which is preferably human.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“PPAR” refers to one or any combination of PPARα, PPARγ and PPARδ.

“PPARγ” refers one or any combination of ΠΠAγ1, PPARγ2, PPARγ3.

“PPAR activator” or “PPARγ activator (agonist)” means any compound that, by any mechanism increases, or causes an increase in the activity of PPARγ or the heterodimer of PPARγ with the retinoid X receptor (RXR), either by direct binding to either PPARγ or RXR or indirectly through any other mechanism that affects the ability of PPARγ or the PPARγ-RXR heterodimer to influence gene expression. Such PPARγ activators may affect PPAR activity either alone or in combination with activation of other PPARs including either PPARα, PPARδ, or both PPARα and PPARδ.

“PPAR-dependent disease” refers to a disease in which 1) administration of a PPAR ligand slows, ameliorates, stops or reverses the pathological process, and/or 2) said disease is associated with impaired signal transduction upstream from PPAR and its interaction with the gene transcription machinery, and/or 3) activation, partial activation or antagonism by a PPAR ligand (PPARα, PPARγ, PPARδ) leads to the prevention, amelioration, cure, or arrest of said disease or pathological process.

“Pre-diabetes” refers to a condition where a patient is pre-disposed to the development of type 2 diabetes. P re-diabetes extends the definition of impaired glucose tolerance to include individuals with a fasting blood glucose within the high normal range≧100 mg/dL (Meigs et al., Diabetes 2003 52:1475-1484) and fasting hyperinsulinemia (elevated plasma insulin concentration).

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Prodrug” refers to a molecule that requires a transformation within the body to release a drug having a specific activity or to be converted to the drug having a specific activity. Prodrugs are frequently, although not necessarily, pharmacologically inactive until converted to the active drug. In some cases a prodrug may have an activity that is different from the activity of the entity into which it is converted. A hydroxyl containing molecule may form, for example, a sulfonate, ester or carbonate prodrug, which may be hydrolyzed in vivo to provide the hydroxyl compound. An amino containing molecule may form, for example, a carbamate, amide, enamine, imine, N-phosphonyl, N-phosphoryl or N-sulfenyl prodrug, which may be hydrolyzed in vivo to provide the amino compound. A carboxylic acid molecule may form an ester (including silyl esters and thioesters), amide or hydrazide prodrug, which may be hydrolyzed in vivo to provide the carboxylic acid compound. Prodrugs for drugs which have functional groups different than those listed above are well known to the skilled artisan.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Substituted” refers to a group in which one or more hydrogen atoms are independently replaced with the same or different substituent(s). Typical substituents include, but are not limited to, those substituents previously defined herein and others. Those include —M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰OR⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is independently a halogen; R⁶⁰, R⁶¹, R⁶² and R⁶³ are independently hydrogen, alkyl, substituted alkyl, alkoxy, substituted alkoxy, cycloalkyl, substituted cycloalkyl, aryl, substituted aryl, cyclic amino, fused 2-, 3-, or 4- ring heterocycle, a 5- or 6-membered heterocyclic ring, or optionally R⁶⁰ and R⁶¹ together with the nitrogen atom to which they are bonded form a cyclic amine ring; and R⁶⁴ and R⁶⁵ are independently hydrogen, alkyl, substituted alkyl, aryl, cycloalkyl, substituted cycloalkyl, substituted aryl, and the like, or optionally R⁶⁴ and R⁶⁵ together with the nitrogen atom to which they are bonded form a cyclic amine ring. Preferably, substituents include —M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O₂)O⁻, —OS(O)₂R⁶⁰, —P(O)(O⁻)₂, —P(O)(OR⁶⁰)(O⁻), —OP(O)(O)(OR⁶⁰)(OR⁶¹), —C(O)R⁶⁰, —C(S)R⁶⁰, —C(O)OR⁶⁰, —C(O)NR^{α}R⁶¹, —C(O)O⁻, —NR⁶²C(O)NR⁶⁰R⁶¹, more preferably, —M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻, most preferably, —M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter) or both. In yet another embodiment, “treating” or “treatment” refers to delaying the onset of the disease or disorder.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated.

“Thiocarboxy” by itself or as part of another substituent refers to the radical —C(O)SR³³ where R³³ is hydrogen, alkyl, cycloalkyl or aryl as defined herein.

“Type 2 diabetes” refers to the condition in which a patient has a fasting blood glucose or serum glucose concentration greater than 125 mg/dl (6.94 mmol/L).

“Visceral obesity” refers to a waist to hip ration of 1.0 in male patients and 0.8 in female patients. In another aspect, visceral obesity defines the risk for insulin resistance and the development of pre-diabetes.

“Weight gain” refers to the increase in body weight in relationship to behavioral habits or addictions, e.g., overeating or gluttony, smoking cessation, or in relationship to biological (life) changes, e.g., weight gain associated with aging in men and menopause in women or weight gain after pregnancy.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### 4.2 Compounds

One aspect of the invention provides compounds of Formula I, which have the following structure: 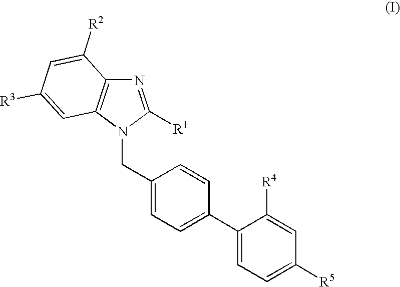
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical, and R⁴ and R⁵ independently are hydrogen, cyanate, or a negatively charged group.
   In preferred aspects of the invention, the compound at least partially activates a PPAR, especially PPARγ. The compound may also inhibit the activity of angiotensin II type I receptor (AT1).

In a preferred embodiment of Formula I, R¹ is hydrogen, halogen, methoxy, hydroxyl, methyl, ethyl, or NH₂, R² is lower alkyl, R³ is phenyl, a fused 2-membered heterocyclic radical or 5- or 6-membered heterocyclic ring, R⁴ is a neutral group, and R⁵ is a negatively charged group. More preferably, R¹ is hydrogen or methyl, R² is lower alkyl, R³ is benzimidazole, R⁴ is a hydrogen, and R⁵ is alkoxycarbonyl or carboxyl.

In yet another preferred embodiment of Formula I, R³ is: 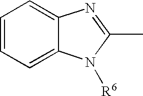
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl. Preferably, R⁶ is hydrogen or lower alkyl.

In yet another preferred embodiment, R¹ is alkyl of 3 or more carbons, R² is hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical, R³ is aryl, 5- or 6-membered heterocyclic ring, or a fused 2-, 3-, or 4-membered heterocyclic radical, R⁴ is a negatively charged group and R⁵ is negatively charged. More preferably, R¹ is propyl or butyl, R² is lower alkyl, R³ is phenyl, a fused 2-membered heterocyclic radical or 5- or 6-membered heterocyclic ring, R⁴ is cyanate, 2-tetrazolyl, carboxyl, alkoxycarbonyl, carbamoyl, sulfonamido, or alkylsulfonamido, and R⁵ is cyanate, 2-tetrazolyl, carboxyl, alkoxycarbonyl, carbamoyl, sulfonamido, or alkylsulfonamido. Even more preferably R¹ is propyl or butyl, R² is lower alkyl, R³ is benzimidazole, R⁴ is lower alkoxycarbonyl or carboxyl, and R⁵ is lower alkoxycarbonyl or carboxyl.

In a preferred embodiment, R¹ is n-propyl, R² is methyl, R³ is 2-(N-Methylbenzimidazolyl), R⁴ and R⁵ are carboxyl, where the compound is:

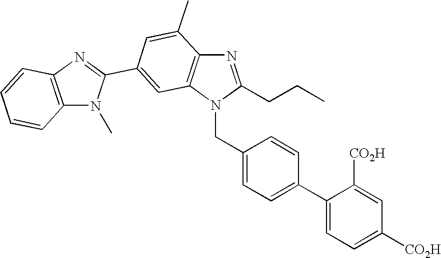

In another preferred embodiment of Formula 1 R¹ is n-propyl, R² is methyl, R³ is 2-(N-Methylbenzoimidazolyl), R⁴ and R⁵ are CO₂Bu^{t}, where the compound is:

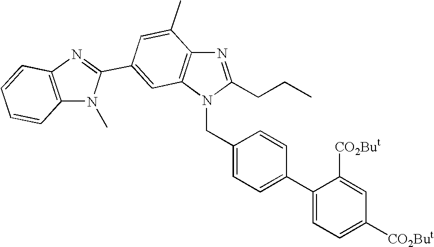

Another aspect of the invention provides compounds of Formula II, which have the following structure: 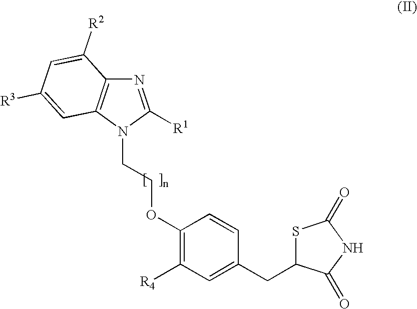
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
n is 0 to 2; and
R⁴ is hydrogen, cyanate, 2-tetrazolyl, carboxyl, alkoxycarbonyl, amido, or sulfonamido.

In a preferred embodiment of Formula II, R¹ is hydrogen, lower alkyl or cyclolower alkylalkyl, R² is hydrogen, lower alkyl, halogen, hydroxyl or NH₂, R³ is phenyl, halogen, hydrogen, amino, alkoxy, hydroxyl, 5- or 6-membered heterocyclic ring, or a fused 2-4-membered heterocyclic radical, and R⁴ is hydrogen, cyantate or a negatively charged group. More preferably, n is 1, R¹ is hydrogen, methyl, ethyl or 4-cyclohexylbutyl, R² is hydrogen, or methyl, R³ is phenyl, halogen, or benzimidazole, and R⁴ is loweralkoxycarbonyl or carboxyl.

In another preferred embodiment of Formula II, R³ is 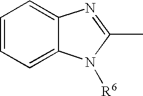
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl. More preferably, R⁶ is hydrogen or lower alkyl.

In a more particular embodiment of Formula II, R¹ is n-propyl, R² is methyl, R³ is 2-(N-methylbenzoimidazolyl), n is one, and R⁴ is methoxycarbonyl, where the compound is:

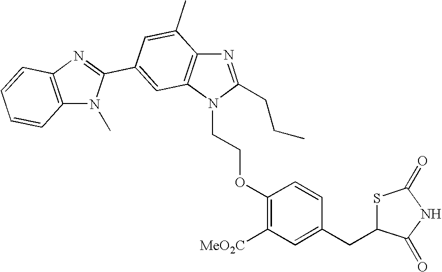

In another embodiment of Formula II, R¹ is n-propyl, R² is methyl, R³ is phenyl, n is one, and R⁴ is methoxycarbonyl, where the compound is:

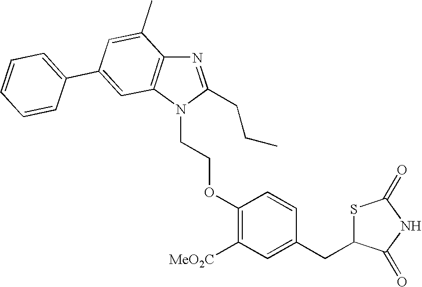

In yet another embodiment of Formula II, R¹ is 4-cyclohexybutyl, R² is hydrogen, R³ is hydrogen, n is one, and R⁴ is methoxycarbonyl, where the compound is:

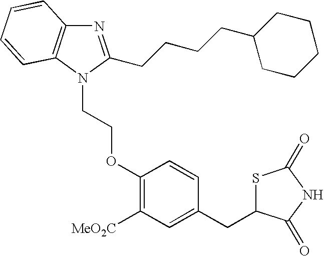

### 4.3 Methods of Synthesis of Compounds of Formulae I and II

The compounds may be obtained via conventional synthetic methods illustrated in Schemes 1-7. Starting materials useful for preparing compounds of the invention and/or intermediates thereof are commercially available or can be prepared by well-known synthetic methods.

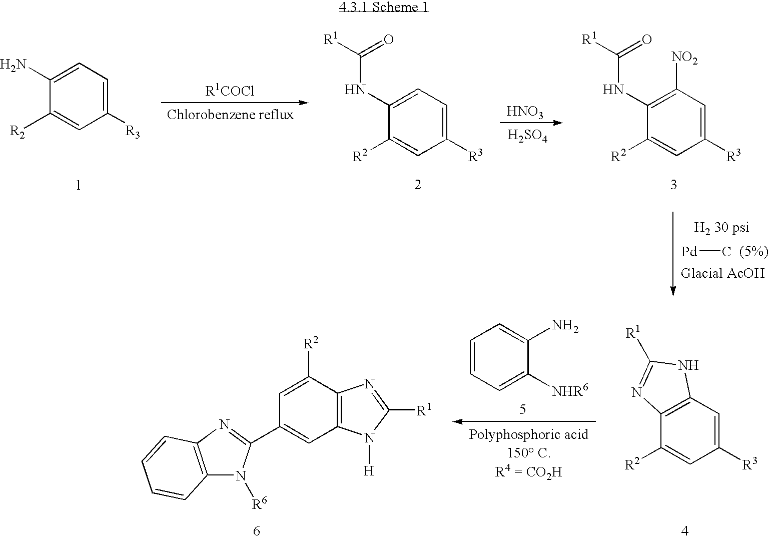

Amino alkylcarboxylates such as 1, wherein R³ can correspond to a great variety of functionality, can be readily N-acylated to furnish anilides 2. Nitration is conveniently ortho-directed by the amide N, while ortho nitration next to the carboxylate group is highly disfavored. The nitro species 3 is then reduced, conveniently by hydrogenation, or also by metal reduction (e.g., Zn, HOAc; Sn, HCl) to afford the amine. Cyclization may occur under the conditions of the reaction, or if necessary, acetic acid can catalyze the cyclization to furnish the benzimidazole 4. De-esterification can be accomplished by saponification with aqueous hydroxide, and the condensation with polyphosphoric acid (PPA) at higher temperatures in the presence of a orthophenylene diamine such as 5, affords the desired biaryl compounds 6 (Zubarovskii, V. M.; Makovetskii, Yu. P. Derivatives of benzimidazolylbenzimidazole. Ukrainskii Khimicheskii Zhurnal (Russian Edition) 1968, 34(11), 1151-1155. Chem. Abstracts: 70: 68251; 2-Aralkyl-5-Arylbenzimidazoles. Chimetron S.a r.l. 1966, 6 pp. FR 1450560 (Patent written in French). Chem. Abstracts: 66: 76010; Schneider, H.; Regioselective nitration of aniline derivatives and benzimidazoles therefrom. 2000, DE 19917524, 6 pp. Chem. Abstracts: 133:298017).

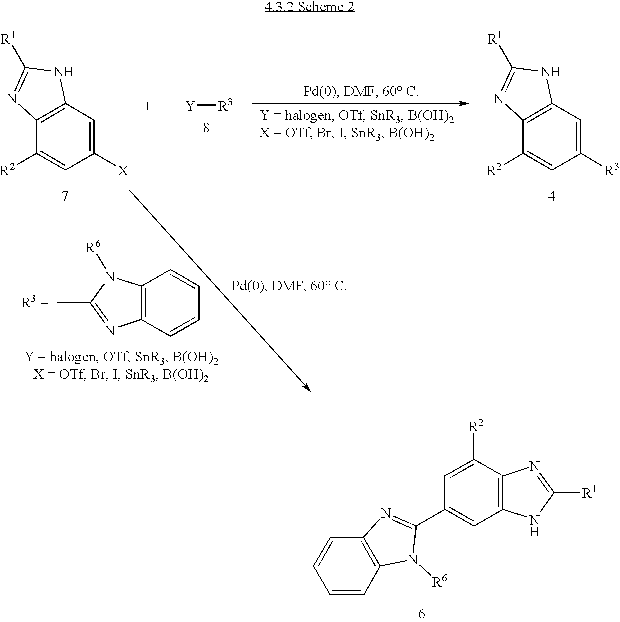

Another approach to the construction of the biaryl systems 6 could involve biaryl coupling chemistry via a 2-halobenzimidazole with a 6-activated benzimidazole as shown in Scheme 2. The benzo-ring activated component, compound 7, can have X=halogen (preferably), OTf, alkyltin, or boronic acid.

Alternatively, the Sn or B derivatives may need to be synthesized from either the OTf or Halogen derivatives if the appropriate starting materials are not practical or readily available. Assuming X is bromo, and then its synthesis should follow the outline in Scheme 2. Another benzimidazole, unsubstituted at C-2, can then be halogenated directly, or lithiated and captured with halogen, Sn or B derivatives. With the two benzimidazoles suitably activated (7 and 8), a Pd(0) catalyzed coupling should afford the same desired compounds outlined in Scheme 1.

In certain cases, 7 may be coupled to provide examples wherein the second ring is not another benzimidazole, but any other substituted heterocycle, alkyl or aryl moiety, or other functionality [Ries, U.; Binder, K.; Zimmermann, R., “Preparation of disubstituted bicyclic heterocyclic antithrombotics and anticoagulants” Ger. Offen. (1998), 44 pp DE 19718181]. Hence, if 7 were coupled with phenylboronic acid, then 4, a simplified version of 6 would be produced as shown in Scheme 2.

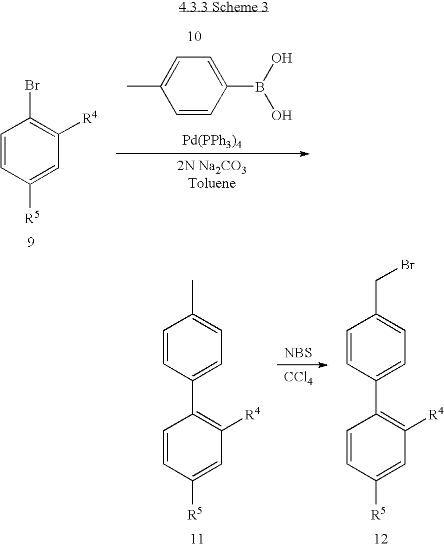

For the construction of the biaryl systems 11 could involve biaryl coupling chemistry via a paratolulyl boronic acid with a halo 2,4-disubstituted benzenes as shown in Scheme 3. The benzo-ring activated component, compound 9, can have X=halogen (preferably), OTf or alkyltin. Benzylic halogenation with N-halo succinamide in the presence of radical initiator such as azobisisobutyronitrile (AIBN) or benzoylperoxide could provide compound 12.

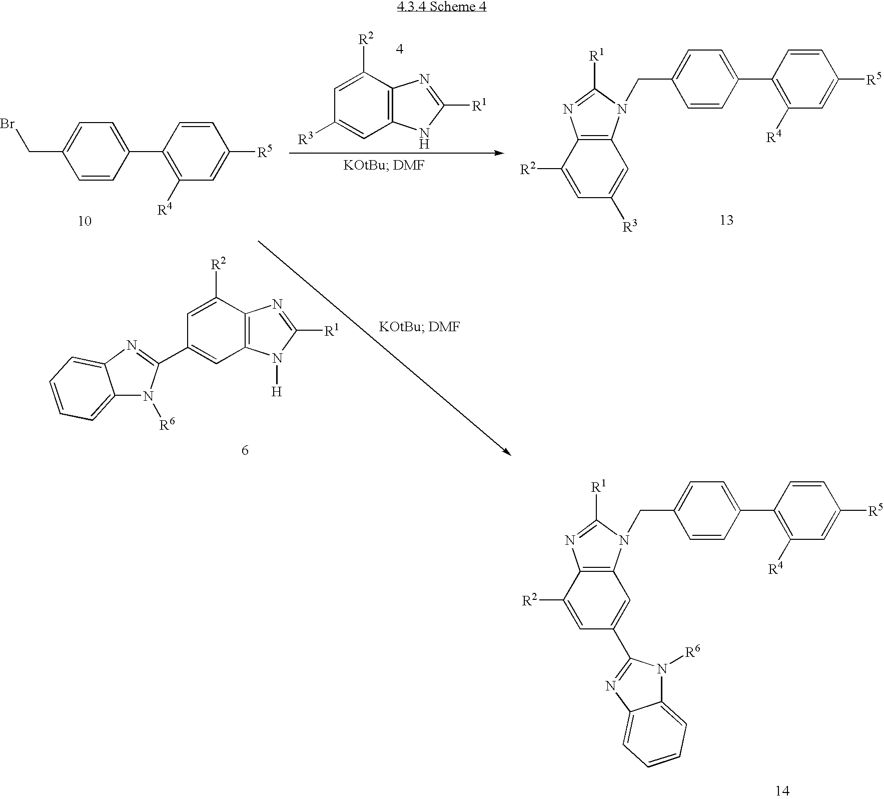

The synthesis of target compounds of Formula I is a variation of the approach used to prepare telmisartan 16, also shown in Scheme 1 (Ries et al., Journal of Medicinal Chemistry 1993, 36, 4040-4051; Hauel, Eur. Pat. Appl.; (Thomae, Dr. Karl, G.m.b.H., Germany). Ep, 1993; pp 25 pp; Hauel, Ger. Offen.; (Thomae, Dr. Karl, G.m.b.H., Germany). De, 1994; pp 23 pp.). Beginning with the reported 2-propyl-4,2′-dimethylbenzimidazolylbenzimidazole 6, N-alkylation of the available benzimidazole ring nitrogen with the bromo-benzylbiaryl derivative 10 proceeds via the predominant/more reactive N-K tautomer (N-1 vs N-3) to afford 14. Beginning with the more general benzimidazole derivative 4, N-alkylation of the available benzimidazole ring nitrogen with the bromo-benzylbiaryl derivative 10 proceed via the predominant/more reactive N-K tautomer to afford 13.

Now, carboxylic acid groups at R⁴ and R⁵ can be exposed by hydrolysis of the ester group under aqueous basic conditions. As stated above, the 2-propyl-4,2′-dimethylbenzimidazolyl benzimidazole 6 can be synthesized, as shown in Schemes 1 or 2, in a manner analogous to that shown to afford general structures 14. Furthermore, throughout these syntheses, cyano groups can often substitute for ester moieties at R⁴ and R⁵ on the bromo benzylbiaryl unit.

Exemplary derivatives of compound 14

| R¹ | R² | R⁴ | R⁵ | R⁶ | Compound |
|---|---|---|---|---|---|
| n-propyl | methyl | CO₂Me | hydrogen | methyl | 15 |
| n-propyl | methyl | CO₂H | hydrogen | methyl | 16 (telmisartan) |
| methyl | methyl | CO₂Me | hydrogen | methyl | 17 |
| methyl | methyl | CO₂H | hydrogen | methyl | 18 |
| n-propyl | methyl | CO₂Me | CO₂Me | methyl | 19 |
| n-propyl | methyl | CO₂H | CO₂H | methyl | 20 |
| methyl | methyl | CO₂Me | CO₂Me | methyl | 21 |
| methyl | methyl | CO₂H | CO₂H | methyl | 22 (avery- |
| | | | | | sartan) |
| hydrogen | hydrogen | CO₂Me | hydrogen | hydrogen | 23 |
| hydrogen | hydrogen | CO₂H | hydrogen | hydrogen | 24 |
| hydrogen | methyl | CO₂Me | hydrogen | methyl | 25 |
| hydrogen | methyl | CO₂H | hydrogen | methyl | 26 |

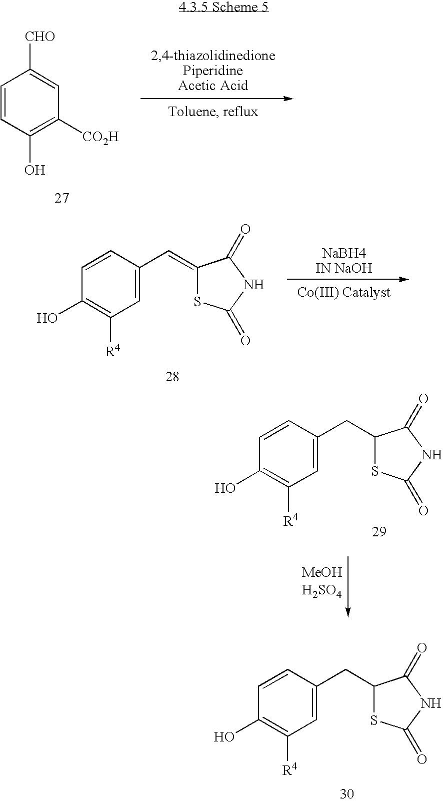

For the preparation of compound 26 could involve Knoveneagel condensation of 2,4-thiazolidinedione with 5-formyl salicylic acid 27 in the presence of catalytic amount of piperidinylacetate. Cobalt (III) chloride catalyzed regiospecific reduction of the 5-benzylidene-2,4-thiazolidinediones using sodium borohydride (Ohnota, M.; Orita, K.; Aizawa, Y.; Yoshida, N.; Sakamaki, T., “Process for the preparation of thiazolidinedione derivatives” PCT Int. Appl. 2001, 17 pp. WO 2001096321) and esterification of the corresponding benzoic acid could provide compound 30.

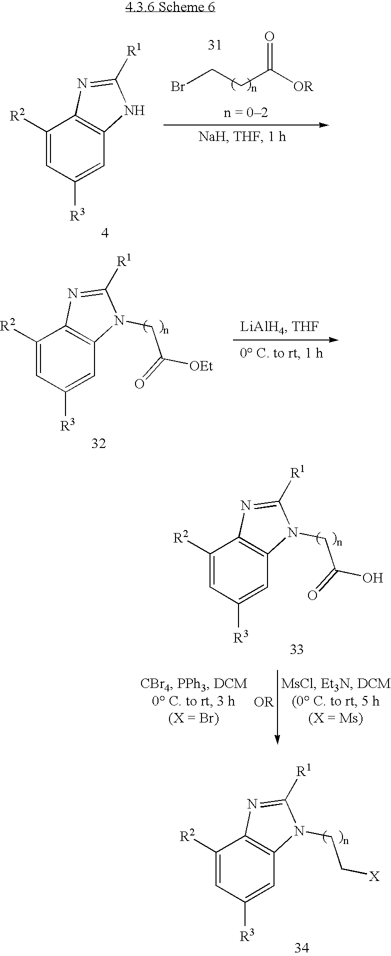

Benzimidazoles such as 4, wherein R₁, R₂, R₃ can correspond to a great variety of functionality, can be readily N-alkylated with bromo esters to furnish 32 [Shen, T-Y.; Dom, C. P., Jr.; Grenda, V. J., “Antiviral 1,2-di-2-benzimidazolyl-1,2-ethanediols” Ger. Offen. (1971), DE 2038952]. The ester species is then reduced conveniently by metal hydride to afford alcohol 33. The hydroxy group could be converted to a better leaving group such as bromide or mesylate 34.

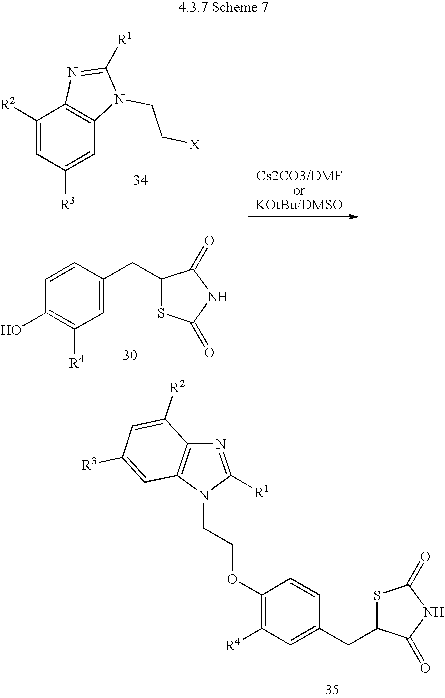

Heterocyclic derivatives of Formula II could arise from simply coupling 34 with the appropriate heterocyclic phenol derivatives 30. Attachment of these side-chains via the appropriate heterocyclic phenol using Cs₂CO₃/DMF or KOtBu/DMSO provides the protected version of the target drugs 35. Either conversion to pro-drug then ensues, or cleavage of the ester.

| R¹ | R² | R³ | R⁴ | Cmpd |
|---|---|---|---|---|
| n-propyl | methyl | N- | CO₂Me | 36 |
| | | methylbenzoimidazolyl | | |
| n-propyl | methyl | phenyl | CO₂Me | 37 |
| n-propyl | methyl | bromo | CO₂Me | 38 |
| n-propyl | methyl | bromo | CO₂Me | 39 |
| 4- | hydro- | hydrogen | CO₂Me | 40 |
| cyclohexylbutyl | gen | | | |

### 4.4 Therapeutic Methods of Use

One aspect of the invention provides methods for treating or preventing an inflammatory or metabolic disorder in a mammal comprising administering to the mammal in need thereof, a therapeutically effective amount of a compound sufficient to at least partially activate a peroxisome proliferator-activated receptor (PPAR).

In one embodiment, a compound of Formulae I or II and/or a pharmaceutical composition thereof is administered to a patient, preferably, a human, suffering from a disease listed in Tables I-X, infra. In another embodiment, a compound of Formulae I or II and/or pharmaceutical composition thereof is administered to a patient, preferably, a human, as a preventative measure against a disease listed in Tables I-X, infra.

TABLE I
Examples of dermatological disorders and inflammatory skin
disorders treatable using compounds of this invention

```
Kertinizing skin diseases, keratitis, hidradenitis, ichthyosis, melasma
Psoriasis (including p. vulgaris, p. guttata, p. discoidea, p. anthropica,
p. universalis)
Acne (including a. vulgaris, a. rosacea, a. inversa, cystic acne)
Warts, verrucae (common warts, anogenital (venereal) warts, viral warts
    including human papilloma virus (HPV) infections, conjunctival
    warts, oral/buccal warts)
Acute and chronic dermatitides (inflammation of the skin), atopic
dermatitis, allergic dermatitis, contact dermatitis, cosmetic dermatitis,
    chemical dermatitis, seborrheic dermatitis, solar dermatitis, acute and
    chronic eczema, diaper rash, sunburn
Lupus associated skin lesions
Keratoses such as seborrheic keratosis, senile keratosis, actinic keratosis,
    photo-induced keratosis, skin aging, thinning skin, dry skin, wrinkle
    formation, photo-induced skin aging, keratosis follicularis
Keloids and prophylaxis against keloid formation
Leukoplakia, lichen planus
Urticaria, pruritus
Androgenic alopecia in men and women, hirsutism in women
```

TABLE II
Examples of psychiatric disorders treatable using
compounds described in this invention

Depression, primary depression, depression secondary to chronic diseases,
medications
Dysphoric mood disorders
Obsessive compulsive disorder
Dysthymic disorders
Manic depressive (unipolar or bipolar) disorder
Anxiety, panic disorder, agoraphobia
Post menstrual syndrome
Schizophrenia
Chronic fatigue syndrome
Substance abuse, drug addiction
Anorexia nervosa, anorexia bullemia

TABLE III
Examples of neurological/neurodegenerative disorders
and CNS inflammatory disorders treatable using
compounds described in this invention

```
Migraine headaches (e.g., vascular headaches, common migraine)
Primary (e.g., Alzheimer's disease) and secondary (e.g., HIV-related)
    dementias
Degenerative CNS diseases (e.g., Parkinson's disease, amyotropic
    lateral sclerosis)
Demyelinating diseases (e.g., multiple sclerosis, Guillain-Barre syndrome)
Pain disorders including algesia, hyperalgesia, acute and chronic
    pain, allodynia
Primary and secondary encephalitis and encephalomyelitis (e.g.,
    autoimmune encephalomyelitis, allergic encephalomyelitis)
Primary and secondary neuritis, autoimmune neuritis
Other autoimmune diseases (e.g., myesthenia gravis, Eaton-Lambert
    syndrome)
Congenital and secondary ataxias
```

TABLE IV
Examples of inflammatory and metabolic disorders
associated with allograft transplantation treatable
using compounds described in this invention

The compounds described herein are useful as monotherapy or adjunctive
therapy with existing immunosuppressive agents for the promotion and
maintenance of allograft survival, post-transplantation.
Examples of inflammatory and proliferative conditions or diseases
associated with allograft transplantation and immune suppression include:

| | |
|---|---|
| 1. | Acute allograft rejection |
| 2. | Chronic allograft rejection |
| 3. | Graft versus host disease |
| 4. | Post-transplantation de novo malignancy (e.g. lymphoma and |
| | epidermal cancers) |
| 5. | Osteoporosis and osteopenia |
| 6. | Hyperlipidemia |
| 7. | Insulin resistance and diabetes mellitus |
| 8. | Hypertension |
| 9. | Atherosclerosis |
| 10. | Endarteritis associated with heart allograft transplantation |
| 11. | Glomerulonephritis associated with renal allograft transplantation |
| 12. | Cardiomyopathy and congestive heart failure associated with allograft |
| | transplantation, in particular heart transplantation |

TABLE V
Examples of diseases of various organ systems treatable using
compounds described in this invention

| Organ System | Disease/Pathology |
|---|---|
| Cardiovascular | Metabolic disorders including hypertension, vasculo- |
| | occlusive diseases including atherosclerosis, arteritis, |
| | endarteritis, endocarditis, myocarditis, arterial plaque |
| | (fibrous cap) rupture, thrombosis, restenosis after any |
| | invasive vascular procedures; acute coronary syndromes |
| | such as unstable angina, myocardial infarction, |
| | myocardial ischemia and other ischemic |
| | cardiomyopathies, non-ischemic cardiomyopathies, |
| | post-myocardial infarction cardiomyopathy and |
| | myocardial fibrosis, drug-induced cardiomyopathy. |
| Endocrine | Metabolic disorders including obesity, type 1 diabetes |
| | mellitus, type 2 diabetes mellitus, gestational diabetes, |
| | impaired glucose tolerance, Cushing's syndrome |
| | (e.g. secondary to chronic glucocorticoid therapy), |
| | polycystic ovarian syndrome, osteoporosis, osteopenia, |
| | accelerated aging of tissues and organs, e.g., Werner's |
| | syndrome. |
| Urogenital | Prostatitis, endometritis, endometriosis, benign prostatic |
| | hypertrophy, leiomyoma, polycystic kidney disease (e.g., |
| | autosomal dominant PKD), acute tubular necrosis, |
| | nephrotic syndrome, diabetic nephropathy, |
| | glomerulonephritis, erectile dysfunction in men |
| | and women |
| Pulmonary | Asthma, chronic obstructive pulmonary disease (COPD), |
| | reactive airway disease, pulmonary fibrosis, pulmonary |
| | hypertension. |
| Connective | Rheumatoid arthritis, Raynaud's phenomenon/disease, |
| tissue | Sjogren's syndrome, systemic sclerosis, systemic |
| Joint | lupus erythematosus, inflammatory bowel disease |
| | (ulcerative colitis, Crohn's disease) vasculitides, |
| | ankylosing spondylitis, osteoarthritis, reactive arthritis, |
| | psoriatic arthritis, fibromyalgia, osteoarthritis, |
| | sarcoidosis. |
| Liver/Other | Hepatic fibrosis, hepatic cirrhosis, hepatic steatosis, all |
| | etiologies, e.g., alcohol-induced (e.g., ethanol), drug- |
| | induced (e.g., tylenol), and toxin-induced (e.g., |
| | mushroom poisoning) Fibrocystic breast disease, |
| | fibroadenoma, endometriosis |

TABLE VIa
Examples of neoplastic diseases treatable using
compounds described in this invention

| Organ System | Malignancy/Cancer type |
|---|---|
| Skin | Basal cell carcinoma, melanoma, squamous cell |
| | carcinoma; cutaneous T cell lymphoma; Kaposi's |
| | sarcoma. |
| Hematological | Acute leukemia, chronic leukemia and myelodysplastic |
| | syndromes. |
| Urogenital | Prostatic, renal and bladder carcinomas, anogenital |
| | carcinomas including cervical, ovarian, uterine, vulvar, |
| | vaginal, and those associated with human papilloma virus |
| | infection. |
| Neurological | Gliomas including glioblastomas, astrocytoma, |
| | ependymoma, medulloblastoma, oligodendroma; |
| | meningioma, pituitary adenoma, neuroblastoma, |
| | craniopharyngioma. |
| Gastrointestinal | Colon, colorectal, gastric, esophageal, mucocutaneous |
| | carcinomas. |
| Breast | Breast cancer including estrogen receptor and |
| | progesterone receptor positive or negative subtypes, |
| | soft tissue tumors. |
| Metastasis | Metastases resulting from all neoplasms. |
| Other | Angiomata, angiogenesis associated with the neoplasms. |

TABLE VIb
Examples of neoplastic diseases treatable using compounds
described in this invention (cont'd)

| Location | Malignancy/Cancer type |
|---|---|
| Various | fibrosarcoma, myxosarcoma, liposarcoma, chondrosarcoma, |
| | osteogenic sarcoma, chordoma, angiosarcoma, |
| | enthotheliosarcoma, lymphangiosarcoma, |
| | lymphangioendotheliosarcoma, synovioma, mesothelimoa, |
| | Ewing's tumor, leiomyosarcoma, rhabdomyosarcoma, |
| | colon carcinoma, basal cell carcinoma, adenocarcinoma, sweat |
| | gland carcinoma, sebaceous gland carcinoma, papillary |
| | carcinoma, papillary adenocarcinomas, cystadenocarcinoma, |
| | medullary carcinoma, bronchogenic carcinoma, renal cell |
| | carcinoma, hepatoma, bile duct carcinoma, choriocarcinoma, |
| | seminoma, embryonal carcinoma, Wilms' tumor, cervical |
| | cancer, testicular tumor, lung carcinoma, small cell lung |
| | carcinoma, bladder carcinoma, epithelial carcinoma, glioma, |
| | astrocytoma, medulloblastoma, craniopharyngioma, |
| | ependymoma, pinealoma, hemangioblastoma, acoustic |
| | neuroma, oligodendroglioma, meningioma, melanoma, |
| | neuroblastoma, and retinoblastoma. |

TABLE VII
Examples of viral infections and related pathologies treatable
according to the methods of this invention

| Virus | Viral infection/cancer or other virus-associated pathology |
|---|---|
| HTLV | T-cell leukemia/lymphoma, HTLV-associated |
| | arthritides/myelopathies. |
| HPV | Cervical and anogenital cancers; common and anogenital |
| | (venereal) warts, including verrucae, condyloma or |
| | condyloma acuminata, related non-neoplastic (e.g., |
| | keratitis, conjunctivitis) pre-neoplastic and neoplastic |
| | (e.g., conjunctival epithelial neoplasms) diseases |
| | of the eye. |
| HAV, HBV, | Hepatitis, hepatocellular carcinoma, lymphoma. |
| HCV | |
| CMV | Hepatitis, retinitis, meningitis. |
| HSV, VSV | Related mucocutaneous, oropharyngeal and genital |
| | diseases, related skin and respiratory infections, varicella- |
| | zoster, chicken pox, herpes zoster, post-herpetic |
| | neuralgia, conjunctivitis, keratoconjunctivitis, keratitis. |
| HHV | Exanthem subitum, infectious mononucleosis. |
| EBV | Infectious mononucleosis, chronic fatigue syndrome, |
| | lymphoma, conjunctivitis, keratitis, and related infections |
| | of the eye. |
| Adenoviruses | Upper and lower respiratory tract infections, pneumonia, |
| | conjunctivitis. |
| RSV | Upper and lower respiratory tract infections, pneumonia. |
| PMV | Mumps and related manifestations, e.g., conjunctivitis. |
| MV, RV | Measles, Rubella (“German measles”) and related |
| | manifestations. |
| Coxsackie | Conjunctivitis, diabetes mellitus, respiratory infections. |
| viruses | |
| Influenza | Upper and lower respiratory tract infections, pneumonia. |
| viruses | |
| HIV, Human Immunodeficiency Virus; HTLV, Human T-cell Lymphocyte Virus; HPV, Human Papilloma Virus; HAV, Hepatitis A Virus; HBV, Hepatitis B Virus; HAV, Hepatitis C Virus; CMV, Cytomegalovirus; HSV, Herpes Simplex Virus | |
| # (Types I & II); HHV, Human Herpes Virus; EBV, Epstein-Barr Virus; RSV, Respiratory Syncytial Virus; VZV, Varicella-Zoster Virus; PMV, Paramyxovirus; MV, Measles (Rubeola) Virus; RV, Rubella | |

TABLE VIII
HIV related infections and diseases treatable using compounds
described in this invention

| | Viral infection/manifestation or other |
|---|---|
| Organ system | HIV-associated disease |
| Immunologic | AIDS, primary HIV infection. |
| Dermatological | Anogenital cancers including rectal and cervical cancer, |
| | Kaposi's sarcoma, atopic dermatitis, squamous cell |
| | carcinoma, hairy leukoplakia, molluscum contagiosum, |
| | warts (HPV infections), seborrheic dermatitis, psoriasis, |
| | xeroderma, HSV and varicella-zoster infections. |
| Hematologic | Non-Hodgkin's lymphoma, B cell lymphoma, anemia, |
| | neutropenia, thrombocytopenia. |
| Gastrointestinal | Anorexia, gastroparesis, diarrhea, malabsorption, |
| | gastrointestinal CMV infections, esophagitis, colitis, |
| | hepatitis, lymphoma. |
| Ophthalmic | Conjunctivitis, keratitis, keratoconjunctivitis, uveitis, |
| | retinitis, chorioretinitis, CMV retinitis, iridocyclitis, |
| | vitreitis, choroiditis, papilledema, Kaposi's sarcoma, |
| | lymphoma, ocular palsies, conjunctival warts, pre- |
| | neoplastic and neoplastic diseases of the eye. |
| Cardiac | Myocarditis, endocarditis, pericarditis. |
| Pulmonary | CMV pneumonitis, lymphoid interstitial pneumonitis. |
| Nephrologic | HIV nephropathy, renal cell carcinoma, amyloidosis, |
| | uropathy. |
| Rheumatologic | Arthralgia, fibromyalgia, Reiter's syndrome, |
| | psoriatic arthritis, vasculitis. |
| Neurologic | Dementia, viral meningitis, viral encephalitis, HIV |
| | encephalopathy, progressive multifocal |
| | leukoencephalopathy, CNS lymphoma, peripheral and |
| | autonomic neuropathies. |
| Psychiatric | Dysphoric mood disorders, depression, depression |
| | associated with chronic diseases and medications, bipolar |
| | disorder, anxiety disorders, chronic fatigue syndrome, |
| | chronic pain, psychoses, substance abuse disorders |
| | and drug addiction. |
| General | Lymphoma, metastatic lymphoma, Kaposi's sarcoma, |
| | wasting syndrome, psychosis. |

TABLE IXa
Diseases of the eye treatable using compounds
described in this invention

| | |
|---|---|
| Disease | Virus |

1. Inflammatory eye diseases associated with viral infections

| | |
|---|---|
| Blepharitis | HSV, VZV, Vaccinia, HPV, molluscum |
| | contagiosum |
| Conjunctivitis | HSV, VZV, EBV, Adenovirus, Vaccinia, Variola, |
| | HPV, molluscum contagiosum, influenza |
| Follicular c. | Newcastle, measles, mumps, rubella, molluscum |
| | contagiosum |
| Hemorrhagic c. | Enterovirus, coxsackie |
| Catarrhal c | Rubella |
| Keratitis | HSV, VZV, EBV, Adenovirus, Vaccinia, Variola, |
| | HPV, molluscum contagiosum |
| Keratoconjunctivitis | HSV, VZV, EBV, Adenovirus, Vaccinia, Variola, |
| | HPV, molluscum contagiosum |
| Retinitis | CMV |
| Uveitis | HPV |
| Conjunctival warts | HPV |
| Epithelial neoplasms | HPV |

2. Oularplastic diseases

| | |
|---|---|
| Benign tumors | Keratocanthoma, molluscum contagiosum, dermoid |
| | cysts, neurofibroma, neurofibromatosis, |
| | schwannoma (neurilemoma), pleiomorphic adenoma |
| Malignant tumors | Basal cell carcinoma, squamous cell carcinoma, |
| | mucoepidermoid carcinoma, melanoma, |
| | retinoblastoma, embryonal rhabdomyosarcoma, |
| | meningioma, adenoid cystic carcinoma, lymphoid |
| | tumors of the orbit, mesenchymal tumors (fibrous |
| | hystiocytoma) of the orbit, nasopharyngeal |
| | carcinoma. |
| Vascular lesions | Hemangioma, lymphangioma |

TABLE XIb
Ophthalmic diseases treatable using compounds
described in this invention (cont'd)
Disease Category/Examples of Diseases,
Causes or Associated Conditions*

| | |
|---|---|
| Conjunctivitis | Acute allergic conjunctivitis (e.g. drug-related |
| | inflammation, hypersensitivity reactions), chronic |
| | (vernal) conjunctivitis, contact lens-associated |
| | conjunctivitis, e.g. giant papillary conjunctivitis, |
| | conjunctival ulceration, including ulceration |
| | associated with mucous membrane, conjunctival |
| | warts |
| Blepharitis | Inflammatory etiologies, e.g. blepharitis secondary |
| | to rosacea |
| Ophthalmic fibrosis | Steven's-Johnson syndrome with progressive |
| | fibrosis and scarring, cicatrization and |
| | symblepharon. |
| Corneal injury | Corneal abrasion or ulceration (e.g. contact lens- |
| | related injury), or corneal injury of any etiology*. |
| Dry eye syndrome | See Table below |
| Pterygium, | |
| pinguecula | |
| Pemphigoid | Includes ophthalmic pemhigori |
| Scleritis/Episcleritis | |
| Iridocyclitis | |
| Endophthalmitis | |
| Uveal tract diseases | Including glaucoma (primary and secondary |
| | etiologies) Uveitis, uveoretinitis, panuveitis |
| Vitreitis, retinitis | e.g. congenital retinitis, retinitis pigmentosa |
| Infectious retinitis | Viral (herpes, cytomegalovirus, HIV), |
| | tuberculous, syphititic, fungal (histoplasmosis) |
| Chorioretinopathies | Chorioretinitis, choroiditis, vitreitis, |
| Retinopathies | Diabetic retinopathy, hypertensive retinopathy |
| Maculopathies | Age-related-macular degeneration, white dot |
| | syndromes |
| Cataract | Related to diabetes, age, collagen vascular |
| Ocular palsies | diseases |

*Etiologies of ophthalmic diseases treatable by compounds of this invention include diseases induced or caused by physical agents (e.g., UV radiation), chemical agents (e.g., acids, caustic solvents) immunological etiologies (e.g., collagen vascular diseases, auto-immune, T lymphocyte-related), infectious agents such as viruses (HSV, CMV, HIV), mycoplasma, tuberculosis, syphilis, fungae (histoplasmosis)

TABLE IXc
Ophthalmic diseases treatable using compounds described in this
invention (cont'd) —Etiologies of dry eye syndrome

```
I. Conditions Characterized by Hypofunction of the Lacrimal Gland:
    A. Congenital
      Familial dysautonomia (Riley-Day syndrome), aplasia of the
      lacrimal gland (congenital alacrima), trigeminal nerve aplasia,
      ectodermal dysplasia
    B. Acquired
      1. Systemic Diseases, e.g. Sjogren's Syndrome, progressive
          systemic sclerosis, sarcoidosis, leukemia, lymphyoma,
          amyloidosis, hemochromatosis,
      2. Infection, e.g. mumps
      3. Injury, e.g. surgical removal of lacrimal gland, irradiation,
          chemical burn
      4. Medications, e.g. antihistamines, antimuscarinics (atropine,
          scopolamine), general anesthetics (halothane, nitrous oxide),
          ∃-adrenergic blockers (timolol, practolol), neurogenic,
          neuroparalytic (facial nerve palsy)
II. Conditions Characterized by Mucin Deficiency
      Avitaminosis A, Stevens-Johnson syndrome, ocular pemphigoid,
      chronic conjuncitivitis (e.g. trachoma), chemical burns, drugs
      and medications
III. Conditions Characterized by Lipid Deficiency
    Lid margin scarring, blepharitis
IV. Defective Spreading of Team Film Caused by eyelid abnormalities:
      1. Defects, colboma
      2. Ectropion or entropion
      3. Keratinization of lid margin
      4. Decreased or absent blinking secondary to: neurologic disorders,
          hyperthyroidism, contact lens, drugs and medications, herpes
          simplex keratitis, leprosy, conjunctival abnormalities,
          pterygium, symblepharon, proptosis
```

TABLE IXd
Ophthalmic diseases treatable using compounds described in this
invention (cont'd) —Non-hereditary and hereditary degenerative diseases

| | |
|---|---|
| Macular | Age-related macular degeneration, exudative macular |
| disorders: | degeneration, atrophic macular degeneration, crystalline |
| | retinopathies, retinal toxicosis of systemic medications, |
| | idiopathic central serous choroidiopathy, macular |
| | edema |
| Retinovascular | Retinopathy, vasculo-occlusive r., ischemic r., |
| diseases and | idiopathic r., hypertensive r., proliferative r., diabetic r., |
| retinopathies: | vitreoretinopathy, vasculopathies associated with |
| | telangiectasias or aneurysms, retinopathies associated |
| | with lupus erythematosus, rheumatoid arthritis, multiple |
| | sclerosis, myasthenia gravis, uveoretinitis or diabetes |
| | mellitus, glaucomatous retinopathies |
| Glaucoma: | Primary and secondary open-angle glaucoma, angle- |
| | closure glaucoma, glaucoma associated with intraocular |
| | inflammation, elevated intraocular pressure associated |
| | with acute glaucoma, steroid-induced glaucoma, |
| | glaucoma associated with intraocular hemorrhage, |
| | pseudoexfoliative syndrome, glaucomatous optic |
| | neuropathy and other degenerative changes (e.g. |
| | retinopathy) associated with glaucoma |
| Cataract: | Age-related (UV radiation) cataract, cataract associated |
| | with systemic diseases such as collagen vascular disease, |
| | diabetes mellitus, Wilson's disease |
| Other diseases: | Primary or secondary retinal detachment |

TABLE IXe

| | |
|---|---|
| 1. | Primary pigmented retinopathies |
| | Autosomal dominant retinitis pigmentosa, e.g. rod-cone and cone-rod |
| | degenerations |
| | Autosomal recessive retinitis pigmentosa, e.g. rod-cone and cone-rod |
| | degenerations, Lerner's amaurosis congenita |
| | X-linked recessive pigmented retinopathies, e.g. choroideremia |
| 2. | Secondary pigmented retinopathies (retinopathies associated with |
| | systemic diseases) |
| | Autosomal dominant pigmented retinopathies, e.g. Paget's disease, |
| | Charcot-Marie-Tooth, disease, Steinert's disease, Pierre-Marie |
| | syndrome |
| | Autosomal recessive pigmented retinopathies, e.g. diabetes mellitus, |
| | mannosidoses, mucopolysccharidoses, Batten's d., Refsum's d., |
| | Usher syndrome |
| | X-linked recessive pigmented retinopathies, e.g. Hunter syndrome |

TABLE X
Diseases or conditions treatable using
compounds described in this invention

I. Promote healing in the following clinical situations:

Surgical or traumatic wounds to healthy tissues or organs
Wounds caused by chemical or physical agents, e.g. ulcers caused by
caustic or erosive chemicals, pressure sores
Wounds associated with disease states, e.g. diabetic ulcers, venous
stasis ulcers
Wounds in diseased tissues or organs

II. Promote cell survival and prevent apoptosis in
neurodegenerative diseases:

Alzheimer's disease
Parkinson's disease
Amyotrophic lateral sclerosis
Spinal cord ischemia, spinal cord injury secondary to trauma or disease

III. Attenuation or arrest of the following conditions or processes:

Time-dependent aging of cells and tissues
Aging induced by chemical or physical agents, e.g. sun-induced skin aging
Accelerated aging associated with diseases, e.g. Werner's syndrome

IV. Vitalization and revitalization of organs and tissues

Promoting cell growth and preventing cell death in the aging process
Promoting therapeutic or non-pathological angiogenesis as a therapeutic
approach to treating diseases such as congestive heart failure and
cardiomyopathy
Promoting growth of organs and tissues for repair or transplantation

The compounds of the instant invention are further useful to suppress the mediators of neurogenic inflammation (e.g., substance P or the tachykinins), and may be used in the treatment of rheumatoid arthritis, psoriasis, topical inflammation such as is associated with sunburn, eczema, or other sources of itching; and allergies, including asthma. The compounds can also function as neuromodulators in the central nervous system, with useful applications in the treatment of Alzheimer's disease and other forms of dementia, pain (as a spinal analgesic), and headaches. Furthermore, in disorders involving myocardial fibrosis, myocardial ischemia, pathological conditions secondary to the autoimmune response to allograft transplantation, the splanchnic blood flow, including hepatic fibrosis, cirrhosis and esophageal varices, the compounds of the invention can provide cytoprotection.

### 4.6 Modes of Administration

The compounds of Formulae I or II and/or pharmaceutical compositions thereof may be advantageously used in human medicine. As previously described, compounds of Formulae I or II and/or pharmaceutical compositions thereof are useful for the treatment or prevention of various diseases listed in Section 4.5.

When used to treat or prevent the above diseases or disorders, compounds and/or pharmaceutical compositions thereof may be administered or applied singly, or in combination with other agents. The compounds and/or pharmaceutical compositions thereof may also be administered or applied singly, in combination with other pharmaceutically active agents including other compounds of Formulae I or II.

The current invention provides methods of treatment and prophylaxis by administration to a patient in need of such treatment of a therapeutically effective amount of a compound and/or pharmaceutical composition thereof. The patient may be an animal, more preferably, is a mammal and most preferably, is a human.

The present compounds and/or pharmaceutical compositions thereof, which comprise one or more compounds of Formulae I and/or II, are preferably administered orally. The compounds and/or pharmaceutical compositions of the invention may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or pharmaceutical composition of the invention. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner and will depend in-part upon the site of the medical condition. In most instances, administration will result in the release of the compounds and/or pharmaceutical compositions of the invention into the bloodstream.

In specific embodiments, it may be desirable to administer one or more compounds and/or pharmaceutical composition of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of the disease.

In certain embodiments, it may be desirable to introduce one or more compounds and/or pharmaceutical compositions of the invention into the central nervous system by any suitable route, including intraventricular, intrathecal and epidural injection. Intraventricular injection may be facilitated by an intraventricular catheter, for example, attached to a reservoir, such as an Ommaya reservoir.

A compound and/or pharmaceutical composition of the invention may also be administered directly to the lung by inhalation. For administration by inhalation, a compound and/or pharmaceutical composition of the invention may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver compounds of the invention directly to the lung.

Alternatively, a Dry Powder Inhaler (“DPI”) device may be used to administer a compound and/or pharmaceutical composition of the invention to the lung. DPI devices typically use a mechanism such as a burst of gas to create a cloud of dry powder inside a container, which may then be inhaled by the patient. DPI devices are also well known in the art. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of a compound of the invention and a suitable powder base such as lactose or starch for these systems.

Another type of device that may be used to deliver a compound and/or pharmaceutical composition of the invention to the lung is a liquid spray device supplied, for example, by Aradigm Corporation, Hayward, Calif. Liquid spray systems use extremely small nozzle holes to aerosolize liquid drug formulations that may then be directly inhaled into the lung.

In one embodiment, a nebulizer is used to deliver a compound and/or pharmaceutical composition of the invention to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96, which is herein incorporated by reference). Examples of nebulizers include devices supplied by Batelle Pulmonary Therapeutics, Columbus Ohio (Armer et al., U.S. Pat. No. 5,954,047; van der Linden et al., U.S. Pat. No. 5,950,619; van der Linden et al., U.S. Pat. No. 5,970,974).

In another embodiment, an electrohydrodynamic (“EHD”) aerosol device is used to deliver a compound and/or pharmaceutical composition of the invention to the lung. EHD aerosol devices use electrical energy to aerosolize liquid drug solutions or suspensions (e.g., Noakes et al., U.S. Pat. No. 4,765,539). The electrochemical properties of the formulation may be important parameters to optimize when delivering a compound and/or pharmaceutical composition of the invention to the lung with an EHD aerosol device and such optimization is routinely performed by one of skill in the art. EHD aerosol devices may more efficiently deliver drugs to the lung than existing pulmonary delivery technologies.

In another embodiment, the compounds and/or pharmaceutical compositions thereof can be delivered in a vesicle, in particular a liposome (Langer, Science, 1990, 249:1527-1533; Treat et al., in “Liposomes in the Therapy of Infectious Disease and Cancer,” Lopez-Berestein and Fidler (eds.), Liss, New York, pp.353-365 (1989))

In another embodiment, the compounds and/or pharmaceutical compositions thereof can be delivered via sustained release systems, preferably oral sustained release systems. In one embodiment, a pump may be used (Langer, supra; Sefton, CRC Crit Ref Biomed Eng. 1987, 14:201; Saudek et al., N. Engl. J Med. 1989, 321:574).

In another embodiment, polymeric materials can be used (Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Langer et al., J Macromol. Sci. Rev. Macromol Chem. 1983, 23:61; Levy et al., Science 1985, 228: 190; During et al., Ann. Neurol. 1989, 25:351; Howard et al., J Neurosurg. 1989, 71:105). In another embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J Pharm. Tech. & Prod. Mfr., 1984, 5(3) 1-9). Factors affecting drug release are well known to the skilled artisan and have been described in the art (Bamba et al., Int. J Pharm., 1979, 2, 307).

In another embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In another embodiment, OROS™ osmotic devices are used for oral sustained release delivery devices (Theeuwes et al., U.S. Pat. No. 3,845,770; Theeuwes et al., U.S. Pat. No. 3,916,899).

In yet another embodiment, a controlled-release system can be placed in proximity of the target of the compounds and/or pharmaceutical composition of the invention, thus requiring only a fraction of the systemic dose (See, e.g., Goodson, in “Medical Applications of Controlled Release,” supra vol. 2, pp. 115-138 (1984)). Other controlled-release systems discussed in Langer, 1990, Science 249:1527-1533 may also be used.

### 4.7 Pharmaceutical Compositions

The present pharmaceutical compositions contain a therapeutically effective amount of one or more compounds of Formulae I and/or II, preferably in purified form, together with a suitable amount of a pharmaceutically acceptable vehicle, so as to provide a form for proper administration to a patient. When administered to a patient, the compounds and pharmaceutically acceptable vehicles are preferably sterile. Water is a preferred vehicle when a compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a compound of Formulae I or II may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds into preparations that can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (Remington, “The Science and Practice of Pharmacy,” Philadelphia College of Pharmacy and Science, 20^{th} Edition, 2000).

For topical administration a compound may be formulated as applicator sticks, solutions, suspensions, gels, creams, ointments, pastes, jellies, paints, powders, aerosols solutions, etc. as is well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In one embodiment, compounds are formulated in accordance with routine procedures as a pharmaceutical composition adapted for intravenous administration to human beings. Typically, compounds for intravenous administration are solutions in sterile isotonic aqueous buffer. For injection, a compound may be formulated in aqueous solutions, preferably, in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent. Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When a compound is administered by infusion, it can be dispensed, for example, with an infusion bottle containing sterile pharmaceutical grade water or saline. When a compound is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optionally agents, for example, sweetening agents such as fructose, aspartame or saccharin; flavoring agents such as peppermint, oil of wintergreen, or cherry coloring agents and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM) etc. Additionally, flavoring agents, preservatives, coloring agents, bile salts, acylcarnitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices will typically include a compound with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspension suitable for use in aerosol devices are known to those of skill in the art (e.g., Biesalski, U.S. Pat. Nos. 5,112,598 and 5,556,611).

A compound may also be formulated in rectal or vaginal pharmaceutical compositions such as suppositories or retention enemas, e.g., containing conventional suppository bases such as cocoa butter or other glycerides.

For ocular administration, a compound may be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and U.S. Pat. No. 4,738,851.

In addition to the formulations described previously, a compound may also be formulated as a depot preparation. Such long acting formulations may be administered by implantation (for example, subcutaneously or intramuscularly) or by intramuscular injection. Thus, for example, a compound may be formulated with suitable polymeric or hydrophobic materials (for example, as an emulsion in an acceptable oil) or ion exchange resins, or as sparingly soluble derivatives, for example, as a sparingly soluble salt.

When a compound is acidic, it may be included in any of the above-described formulations as the free acid, a pharmaceutically acceptable salt, a solvate or a hydrate. Pharmaceutically acceptable salts substantially retain the activity of the free acid, may be prepared by reaction with bases and tend to be more soluble in aqueous and other protic solvents than the corresponding free acid form.

### 4.8 Doses

The present methods for treatment or prevention of the various diseases listed in Tables (I-X), supra, require administration of a compound of Formulae I or II, or a pharmaceutical composition thereof, to a patient in need of such treatment or prevention.

The amount of a compound that will be effective in the treatment or prevention of the various diseases listed in Tables (I-X), supra, in a patient will depend on the specific nature of the condition and can be determined by standard clinical techniques known in the art. In addition, in vitro or in vivo assays may optionally be employed to help identify optimal dosage ranges. The amount of a compound administered will, of course, be dependent on, among other factors, the subject being treated, the weight of the subject, the severity of the affliction, the manner of administration and the judgment of the prescribing physician.

Preferably, the dosage forms are adapted to be administered to a patient no more than twice per day, more preferably, only once per day. Dosing may be provided alone or in combination with other drugs and may continue as long as required for effective treatment or prevention of the various diseases listed in Tables (I-X), supra.

Suitable dosage ranges for oral administration are dependent on the potency of the particular compound but are generally about 0.1 mg to about 200 mg of drug per kilogram body weight, more preferably about 1 to about 100 mg of drug per kilogram body weight per day. Dosage ranges for topical treatment are between about 0.1% to about 0.5% (weight/volume) in a gel, cream or ointment. A typical dose for intra-dermal or intraocular injection is between 0.25 to about 10 mg depending the portion of eye that is being treated. Dosage ranges for other forms of administration may be readily determined by methods known to the skilled artisan.

### 4.9 Combination Therapy

In certain embodiments, a compound of Formula I or II and/or pharmaceutical compositions thereof can be used in combination therapy with at least one other therapeutic agent which may be a different compound of Formula I or II and/or pharmaceutical compositions thereof. The compound and/or pharmaceutical composition thereof and the other therapeutic agent can act additively or, more preferably, synergistically. In one embodiment, a compound and/or a pharmaceutical composition thereof is administered concurrently with the administration of another therapeutic agent. In another embodiment, a compound and/or pharmaceutical composition thereof is administered prior or subsequent to administration of another therapeutic agent.

Examples of other therapeutic agents which can be used with compounds of Formulae I and II include, but are not limited to, diabetes mellitus-treating agents, diabetic complication-treating agents, antihyperlipemic agents, hypotensive or antihypertensive agents, anti-obesity agents, diuretics, chemotherapeutic agents, immunotherapeutic agents immunosuppressive agents, and the like.

Examples of agents for treating diabetes mellitus include insulin formulations (e.g., animal insulin formulations extracted from a pancreas of a cattle or a swine; a human insulin formulation synthesized by a gene engineering technology using microorganisms or methods), insulin sensitivity enhancing agents, pharmaceutically acceptable salts, hydrates, or solvates thereof (e.g., pioglitazone, troglitazone, rosiglitazone, netoglitazone, balaglitazone, rivoglitazone, tesaglitazar, farglitazar, CLX-0921, R-483, NIP-221, NIP-223, DRF-2189, GW-7282TAK-559, T-131, RG-12525, LY-510929, LY-519818, BMS-298585, DRF-2725, GW-1536, GI-262570, KRP-297, TZD18 (Merck), DRF-2655, and the like), alpha-glycosidase inhibitors (e.g., voglibose, acarbose, miglitol, emiglitate and the like), biguanides (e.g., phenformin, metformin, buformin and the like)or sulfonylureas (e.g., tolbutamide, glibenclamide, gliclazide, chlorpropamide, tolazamide, acetohexamide, glyclopyramide, glimepiride and the like) as well as other insulin secretion-promoting agents (e.g., repaglinide, senaglinide, nateglinide, mitiglinide, GLP-1 and the like), amyrin agonist (e.g., pramlintide and the like), phosphotyrosinphosphatase inhibitor (e.g., vanadic acid and the like) and the like.

Examples of agents for treating diabetic complications include, but are not limited to, aldose reductase inhibitors (e.g., tolrestat, epalrestat, zenarestat, zopolrestat, minalrestat, fidareatat, SK-860, CT-112 and the like), neurotrophic factors (e.g., NGF, NT-3, BDNF and the like), PKC inhibitors (e.g., LY-333531 and the like), advanced glycation end-product (AGE) inhibitors (e.g., ALT946, pimagedine, pyradoxamine, phenacylthiazolium bromide (ALT766) and the like), active oxygen quenching agents (e.g., thioctic acid or derivative thereof, a bioflavonoid including flavones, isoflavones, flavonones, procyanidins, anthocyanidins, pycnogenol, lutein, lycopene, vitamins E, coenzymes Q, and the like), cerebrovascular dilating agents (e.g., tiapride, mexiletene and the like).

Antihyperlipemic agents include, for example, statin-based compounds which are cholesterol synthesis inhibitors (e.g., pravastatin, simvastatin, lovastatin, atorvastatin, fluvastatin, rosuvastatin and the like), squalene synthetase inhibitors or fibrate compounds having a triglyceride-lowering effect (e.g., fenofibrate, gemfibrozil, bezafibrate, clofibrate, sinfibrate, clinofibrate and the like).

Hypotensive agents include, for example, angiotensin converting enzyme inhibitors (e.g., captopril, enalapril, delapril, benazepril, cilazapril, enalapril, enalaprilat, fosinopril, lisinopril, moexipril, perindopril, quinapril, ramipril, trandolapril and the like) or angiotensin II antagonists (e.g., losartan, candesartan cilexetil, olmesartan medoxomil, eprosartan, valsartan, telmisartan, irbesartan, tasosartan, pomisartan, ripisartan forasartan, and the like).

Antiobesity agents include, for example, central antiobesity agents (e.g., dexfenfluramine, fenfluramine, phentermine, sibutramine, amfepramone, dexamphetamine, mazindol, phenylpropanolamine, clobenzorex and the like), gastrointestinal lipase inhibitors (e.g., orlistat and the like), β-3 agonists (e.g., CL-316243, SR-58611-A, UL-TG-307, SB-226552, AJ-9677, BMS-196085 and the like), peptide-based appetite-suppressing agents (e.g., leptin, CNTF and the like), cholecystokinin agonists (e.g., lintitript, FPL-15849 and the like) and the like.

Diuretics include, for example, xanthine derivatives (e.g., theobromine sodium salicylate, theobromine calcium salicylate and the like), thiazide formulations (e.g., ethiazide, cyclopenthiazide, trichloromethiazide, hydrochlorothiazide, hydroflumethiazide, bentylhydrochlorothiazide, penflutizide, polythiazide, methyclothiazide and the like), anti-aldosterone formulations (e.g., spironolactone, triamterene and the like), decarboxylase inhibitors (e.g., acetazolamide and the like), a chlorbenzenesulfonamide formulations (e.g., chlorthalidone, mefruside, indapamide and the like), azosemide, isosorbide, ethacrynic acid, piretanide, bumetanide, furosemide and the like.

Chemotherapeutic agents include, for example, alkylating agents (e.g., cyclophosphamide, iphosphamide and the like), metabolism antagonists (e.g., methotrexate, 5-fluorouracil and the like), anticancer antibiotics (e.g., mitomycin, adriamycin and the like), vegetable-derived anticancer agents (e.g., vincristine, vindesine, taxol and the like), cisplatin, carboplatin, etoposide and the like. Among these substances, 5-fluorouracil derivatives such as furtulon and neofurtulon are preferred.

Immunotherapeutic agents include, for example, microorganisms or bacterial components (e.g., muramyl dipeptide derivative, picibanil and the like), polysaccharides having immune potentiating activity (e.g., lentinan, sizofilan, krestin and the like), cytokines obtained by a gene engineering technology (e.g., interferon, interleukin (IL) and the like), colony stimulating factors (e.g., granulocyte colony stimulating factor, erythropoetin and the like) and the like, among these substances, those preferred are IL-1, IL-2, IL-12 and the like.

Immunosuppressive agents include, for example, calcineurin inhibitor/immunophilin modulators such as cyclosporine (Sandimmune, Gengraf, Neoral), tacrolimus (Prograf, FK506), ASM 981, sirolimus (RAPA, rapamycin, Rapamune), or its derivative SDZ-RAD, glucocorticoids (prednisone, prednisolone, methylprednisolone, dexamethasone and the like), purine synthesis inhibitors (mycophenolate mofetil, MMF, CellCept(R), azathioprine, cyclophosphamide), interleukin antagonists (basiliximab, daclizumab, deoxyspergualin), lymphocyte-depleting agents such as antithymocyte globulin (Thymoglobulin, Lymphoglobuline), anti-CD3 antibody (OKT3), and the like.

In addition, agents whose cachexia improving effect has been established in an animal model or at a clinical stage, such as cyclooxygenase inhibitors (e.g., indomethacin and the like) [Cancer Research, Vol.49, page 5935-5939, 1989], progesterone derivatives (e.g., megestrol acetate) [Journal of Clinical Oncology, Vol.12, page 213-225, 1994], glucosteroid (e.g., dexamethasone and the like), metoclopramide-based agents, tetrahydrocannabinol-based agents, lipid metabolism improving agents (e.g., eicosapentanoic acid and the like) [British Journal of Cancer, Vol.68, page 314-318, 1993], growth hormones, IGF-1, antibodies against TNF-.alpha., LIF, IL-6 and oncostatin M may also be employed concomitantly with a compound according to the present invention.

The preferred combinations of the agents for the prevention and/or treatment of diabetes are, a compound according to the current invention and:
1) an insulin formulation and a biguanide;
2) a sulfonylurea agent and a biguanide;
3) a sulfonylurea agent and an alpha-glycosidase inhibitor;
4) a biguanide and an alpha-glycosidase inhibitor;
5) a blood sugar reducing agent and other kind of agents for treating diabetic complications;
6) an 3-hydroxy-3-methylglutaryl coenzyme A (HMG CoA) reductase inhibitor;
7) any other two kinds of agents mentioned above;
8) an agent that inhibits activity of angiotensin converting enzyme;
9) an anti-dyslipidemic fibrate.

The preferred combinations of the agents for the prevention and/or treatment of diabetes are, a compound according to the current invention and:
1) an insulin formulation and a biguanide;
2) a sulfonylurea agent and a biguanide;
3) a sulfonylurea agent and an alpha-glycosidase inhibitor;
4) a biguanide and an alpha-glycosidase inhibitor;
5) a blood sugar reducing agent and the other kind of agents for treating diabetic complications;
6) an 3-hydroxy-3-methylglutaryl coenzyme A (HMG CoA) reductase inhibitor;
7) any other two kinds of agents mentioned above;
8) an agent that inhibits activity of angiotensin converting enzyme;
9) an anti-dyslipidemic fibrate.

In case that a compound or the composition of the present invention is used in combination with the other agent, an amount of each other agent can be reduced in a range which is safe in light of its adverse effect. Especially, an insulin sensitivity enhancing agent, a biguanide and a sulfonylurea agent can be used at a lower dose than those usually used so that adverse effects which may be caused by these agents can be safely avoided. In addition, an agent for treating diabetic complications, an anti-hyperlipemic agent and a hypotensive agent can also be used at a lower dose, so that adverse effect which may be caused by them can be avoided effectively.

Administering both an angiotensin receptor blocker and a compound of the present invention formulated together in a single pill or tablet, may be used to treat glucose intolerance or type 2 diabetes and other PPAR responsive disorders without causing fluid retention, edema, or congestive heart failure. For this purpose, a pharmaceutical composition comprising: (i) a compound of the present invention in a therapeutically effective amount sufficient to prophylactically prevent, slow, delay or treat a metabolic, inflammatory, atopic, autoimmune, proliferative, or cardiovascular disorder in humans; (ii) an angiotensin II type 1 receptor antagonist in a therapeutically effective amount sufficient to prevent, slow, delay, or treat fluid retention, peripheral edema, pulmonary edema, or congestive heart failure; and (iii) a pharmaceutically acceptable vehicle may be formulated. The angiotensin II type 1 receptor antagonist may be a compound selected from the group consisting of telmisartan, irbesartan, valsartan, losartan, candesartan, candesartan cilexetil, olmesartan, olmesartan medoximil, losartan, valsartan, eprosartan, irbesartan, tasosartan, pomisartan, ripisartan, and forasartan, or an analog thereof, or a tautomeric form thereof, or a pharmaceutically acceptable salt thereof, or a pharmaceutically acceptable solvate thereof.

It should also be noted the compounds of the present invention may be administered with a thiazolidinedione selected from the group consisting of pioglitazone, troglitazone, rosiglitazone, netoglitazone, balaglitazone, rivoglitazone, CLX-0921, R-483, NIP-221, NIP-223, DRF-2189, and the like, or an analog thereof, or a tautomeric form thereof, or a pharmaceutically acceptable salt thereof, or a pharmaceutically acceptable solvate thereof to treat or prevent various disorders listed in Tables (I-X), supra. Alternatively, the compounds of the present invention may be administered with a non-thiazolidinedione selected from the group of compounds consisting of tesaglitazar, farglitazar, ragaglitazar, LY818, T131, LSN862, DRF 4832, LM 4156, LY 510929, LY 519818, TY 51501, X 334, GW-7282, TAK-559, T-131, RG-12525, BMS-298585, DRF-2725, GW-1536, GI-262570, KRP-297, TZD18 (Merck), DRF-2655, or an analog thereof, or a tautomeric form thereof, or a pharmaceutically acceptable salt thereof, or a pharmaceutically acceptable solvate thereof. Other thiazolidinedione or non-thiazolidinedione activators of PPARs that are familiar to those skilled in the art can also be employed in combination with the compound of the invention to treat or prevent various disorders listed in Tables (I-X), supra.

The compounds of this invention can also be given orally in combination with natural or synthetic compounds that bind to or modify the activity of the vitamin D receptor or other nuclear hormone receptor or in combination with compounds that bind to or modify the activity of the retinoid X receptor to provide for a synergistic effect in the treatment or prevention of the disorders listed in Table I-X, supra. Examples of such compounds include, but are not limited to, vitamin D analogs, various retinoic acid derivatives, and other ligands for retinoid X receptors or retinoic acid receptors including but not limited to compounds such as LG100268, tazarotene, TTNPB, AGN 190121, adapalene or LGD1069 (Targretin).

Synergistic therapeutic effects can be achieved by oral or topical administration of the compounds of the current invention together with orally, topically or intravenously administered drugs that bind to and modify the activity of either the vitamin D receptor, the glucocorticoid receptor, the intracellular enzyme calcineurin, the retinoid X receptors, the retinoic acid receptors, or other PPARs such as PPARα or PPARδ. Examples of effective retinoids are 9-cis-retinoic acid, 13-cis-retinoic acid, all-trans-retinoic acid (at-RA). Preferred retinoids for this purpose would include 13-cis-retinoic acid, tazarotene, or Targretin. Examples of effective vitamin D analogs are 1,25-dihydroxy-vitamin D, calcipotriene and calcipotriol.

Synergistic therapeutic effects can be achieved by oral or topical administration of the compounds of the current invention together with orally, topically or intravenously administered natural or synthetic antioxidants. These include, but are not limited to, ascorbic acid and its derivatives (e.g., vitamin C), the tocopherols (e.g., vitamin E, vitamin E succinate), carotenes and carotenoids (e.g., β-carotene), alpha-lipoic acid, probucols, flavones, isoflavones and flavonols (e.g., quercetin, genistein, catechin, apigenin, lutein, luteolin), lycopene, pycnogenol, glutathione and its derivatives (e.g., N-acetylcysteine and dithiothreitol), and phytoestrogens and phenolic anthocyanidin and procyanidin derivatives (e.g., resveratrol, cyanidin, cinnamic acid).

### 4.10 Assays

The compounds of the current invention may be tested for their ability to activate PPARγ isoforms by utilizing standard screening methods known to those skilled in the art including, but not limited to, cell based transactivation assays or cell free assays that test the ability of a compound to activate PPARγ construct by measuring the output of a reporter signal that reflects the extent of the PPAR activation. For example, a compound of the current invention is added to the culture media of CV1 cells or other cells that can be transfected with a full length or partial PPARγ cDNA sequence together with a reporter construct containing a PPAR response element or other appropriate response element fused to a reporter gene such as luciferase. The ability of a compound of the current invention to activate PPARγ is tested by measuring the luciferase reporter gene activity. Any compound found to activate PPARγ according to these or other methods can be used to treat disorders known to be responsive to PPAR activators.

PPARγ activators that have improved safety profile and decreased risk for causing fluid retention, edema, or congestive heart failure may be identified by testing their ability to inhibit angiotensin converting enzyme activity or their ability to block the angiotensin receptor. PPARγ ligands or PPARγ activators that also inhibit ACE activity or block angiotensin II type 1 receptors represent an improvement over existing PPAR ligands for treating PPAR responsive disorders because they have reduced likelihood of causing fluid retention, edema, or congestive heart failure. A variety of assays are available that can be used by those skilled in the art to determine whether a PPARγ activator can also block the angiotensin II type 1 receptor or inhibit the activity of angiotensin converting enzyme.

### 5. Examples

The invention is further defined by reference to the following examples. It will be apparent to those skilled in the art that many modifications, both to materials and methods, may be practiced without departing from the scope of the invention.

### 5.1 Example 1

### Suzuki coupling of 4-tolylboronic acid with dimethyl 4-bromobenzene-1,3-dioate

To a round bottom flask equipped with a magnetic stir bar, heating mantle, and reflux condenser was added bromo compound, dimethyl (4′-methylphenyl)benzene-1,3-dioate, (1 mmol), 4-tolylboronic acid (1.1 mmol), toluene (21 mL), 2N sodium carbonate solution (6 mL), methanol 3(mL) and tetrakistriphenyl phosphine (5% mol). The resulting mixture was vigorously refluxed until TLC showed disappearance of bromophenyl derivative (˜2 h). After completion of the reaction, the reaction mixture was cooled, neutralized with 2N HCl and extracted with EtOAc. The combined organic layers were washed with water, brine and dried over anhydrous MgSO₄ and filtered. The filtrate was concentrated under vacuum, purification by column chromatography afforded the biphenyl derivative, dimethyl (4′-methylphenyl)benzene-1,3-dioate (yield 91%).

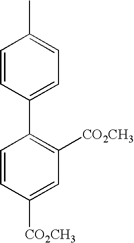

¹HNMR (CDCl₃, 400 MHz): δ 2.40 (s, 3H); 3.71 (s, 3H); 3.95 (s, 3H); 7.22 (s, 4H); 7.45 (d, 1H); 8.16 (d, 1H); 8.46 (s, 1H)

### 5.2 Example 2

### Bromination with NBS

A solution of dimethyl (4′-methylphenyl)benzene-1,3-dioate (10 mmol), N-bromosuccinamide (12 mmol) and benzoylperoxide (0.1 mmol) in carbon tetrachloride (25 mL) was refluxed for 3 h. After cooling, the mixture was filtered, and the filtrate was concentrated under vacuum to give a residue, dimethyl (4′-bromomethyl)benzene-1,3-dioate, that was purified by column chromatography (yield 85%).

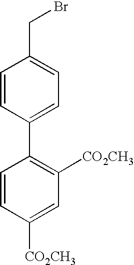

¹HNMR (CDCl₃, 400 MHz): δ 3.47 (s, 2H); 3.66 (s, 3H); 3.95 (s, 3H); 7.26 (d, 2H); 7.35 (d, 2H); 7.46 (d, 1H); 8.16 (dd, 1H); 8.46 (d, 1H).

### 5.3 Example 3

### Preparation of (Z)-5-((2,4-dioxothiazolidin-5-ylidene)methyl)-2-hydroxybenzoic acid

A mixture of the 5-formyl-2-hydroxybenzoic acid (6.64 g, 40 mmol), 2,4-thiazolidinedione (4.68 g, 40 mmol), piperidine (0.085 g, 1 mmol) and acetic acid (0.06 g, 1 mmol) in toluene (40 mL) was heated under reflux with azeotropic removal of water for 12 h. The mixture was cooled to 5° C. Filtration gave a pale orange solid, 2-hydroxy-5-((Z)-(2,4-dioxothiazolidin-5-ylidene)methyl)benzoic acid, which was washed with cold toluene and dried (9.22 g, yield 87%).

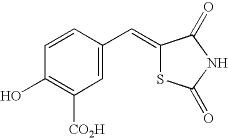

¹HNMR (400 MHz, CDCl₃): δ 6.90 (d, 1H); 7.56 (d, 1H); 7.69 (s, 1H); 7.96 (s, 1H); 8.46 (bs, 1H); 12.4 (bs, 1H).

### 5.4 Example 4

### Preparation of methyl 2-hydroxy-5-((2,4-dioxothiazolidin-5-yl)methyl)benzoate

A solution of enone, (2-hydroxy-5-((Z)-(2,4-dioxothiazolidin-5-ylidene)methyl)benzoic acid, (2.65 g, 10 mmol) in 1M aq NaOH (20 mL) was adjusted to pH 9.1 by addition of 1M aq. HCl, was successively treated with a solution of chloro(pyridine)bis(dimethylglyoximato)cobalt(III) (40 mg, 0.1 mmol) in water (1 mL) and sodium borohydride (0.25 g, 6.5 mmol) and stirred at 30° C. After 2 h, the reaction mixture was cooled to 20-25° C., and (the pH of the reaction mixture) adjusted to 2.0 by addition of 1M aq. HCl. After extraction with EtOAc, the combined organic layers were washed with brine, dried over anhydrous Na₂SO₄ and concentrated under vacuum. The crude acid was converted to methyl ester, methyl 2-hydroxy-5-((2,4-dioxothiazolidin-5-yl)methyl)benzoate, by dissolving in anhydrous methanol (20 mL) with catalytic conc. H₂SO₄ (2.1 g, 74% two steps).

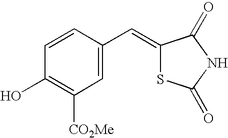

¹HNMR (CDCl₃, 400 MHz): δ 3.11 (dd, 1H); 3.43 (dd, 1H); 3.95 (s, 3H); 4.51 (dd, 1H); 6.95 (d, 1H); 7.32 (d, 1H); 7.70 (s, 1H); 8.78 (bs, 1H); 10.73 (s, 1H).

### 5.5 Example 5

### Preparation of methyl 7-methyl-2-propyl-3H-benzo[d]imidazole-5-carboxylate

Methyl 4-amino-3-methyl-5-nitrobenzoate (10 mmol) was acylated with butyryl chloride (10 mmol) in chlorobenzene at 100° C. After completion of the reaction the reaction mixture was washed with saturated sodium bicarbonate and extracted with chlorobenzene. The resulting amide was reacted with fuming nitric acid in sulfuric acid at 0° C. The resulting nitro-amide was reduced with hydrogen (30 psi) and palladium (5%) on charcoal in methanol. After completion of the reaction, the reaction mixture was filtered through Celite, and the filtrate was evaporated in vacuo (yield 54%).
Methyl 4-(butyramido)-3-methyl-5-nitrobenzoate:

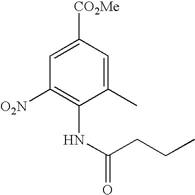

¹HNMR (CDCl₃, 400 MHz,): δ 1.02 (t, 3H); 1.79 (m, 2H); 2.30 (s, 3H); 2.43 (t, 2H); 3.90 (s, 3H); 5.72 (bs, 1H); 8.12 (s, 1H); 8.47 (s, 1H)

The crude product, methyl 3-amino-4-(butyramido)-5-methylbenzoate, was dissolved in glacial acetic acid (20 mL) and heated to reflux for 2 h. After evaporation, water (50 mL) was added, the pH was adjusted to 8 by addition of concentrated ammonia, and the mixture was extracted with ethyl acetate. The combined extracts were dried, the solvent was evaporated, and the residue was purified by column chromatography gave methyl 7-methyl-2-propyl-3H-benzo[d]imidazole-5-carboxylate (yield 88%).

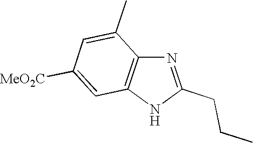

¹HNMR (CDCl₃, 400 MHz,): δ 0.97 (t, 3H); 1.88 (m, 2H); 2.58 (s, 3H); 2.94 (t, 2H); 3.93 (s, 3H); 7.78 (s, 1H); 8.12 (s, 1H)

### 5.6 Example 6

### Preparation of 6-bromo4-methyl-2-propyl-1H-benzo[d]imidazole

4-Bromo-2-methyl-6-nitrobenzenamine (10 mmol) was acylated with butyryl chloride (10 mmol) in chlorobenzene at 100° C. After completion of the reaction the reaction mixture was washed with saturated sodium bicarbonate and extracted with chlorobenzene. The resulting amide was reacted with fuming nitric acid in sulfuric acid at 0° C. The resulting nitro-amide was reduced with hydrogen (30 psi) and palladium (5%) on charcoal in methanol. After completion of the reaction, the reaction mixture was filtered through Celite, and the filtrate was evaporated in vacuo (yield 65%).
N-(4-bromo-2-methyl-6-nitrophenyl)butyramide:

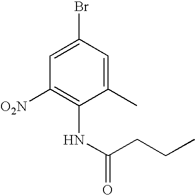

¹HNMR (CDCl₃, 400 MHz,): δ 0.90 (t, 3H); 1.64 (m, 2H); 2.22 (s, 3H); 2.45 (t, 2H); 7.75 (s, 1H); 8.06 (s, 1H)

The crude product, N-(2-amino-4-bromo-6-methylphenyl)butyramide, was dissolved in glacial acetic acid (20 mL) and heated to reflux for 2 h. After evaporation, water (50 mL) was added, the pH was adjusted to 8 by addition of concentrated ammonia, and the mixture was extracted with ethyl acetate. The combined extracts were dried, the solvent was evaporated, and the residue was purified by column chromatography gave 6-Bromo-4-methyl-2-propyl-1H-benzo[d]imidazole (yield 76%).

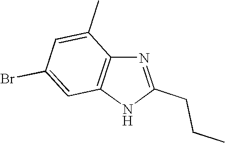

¹HNMR(CDCl₃, 400 MHz,): δ 0.97 (t, 3H); 1.86 (m, 2H); 2.52 (s, 3H); 3.07 (t, 2H); 7.34 (m, 5H); 7.39 (d, 1H); 7.49 (s, 1H)

### 5.7 Example 7

### Preparation of 4-methyl-6-phenyl-2-propyl-1H-benzo[d]imidazole

To a round bottom flask equipped with a magnetic stir bar, heating mantle, and reflux condenser was added 6-Bromo-4-methyl-2-propyl-1H-benzo[d]imidazole (example 6) (1 mmol), phenylboronic acid (1.1 mmol), toluene (21 mL), 2N sodium carbonate solution (6 mL), methanol 3(mL) and tetrakistriphenyl phosphine (5% mol). The resulting mixture was vigorously refluxed until TLC showed disappearance of 6-Bromo-4-methyl-2-propyl-1H-benzo[d]imidazole (˜3 h). After completion of the reaction, the reaction mixture was cooled, neutralized with 2N HCl and extracted with EtOAc. The combined organic layers were washed with water, brine and dried over anhydrous MgSO₄ and filtered. The filtrate was concentrated under vacuum, purification by column chromatography afforded the biphenyl derivative, 4-methyl-6-phenyl-2-propyl-1H-benzo[d]imidazole (yield 91%).

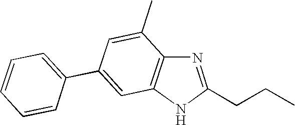

¹HNMR(CDCl₃, 400 MHz,): δ 0.92 (t, 3H); 1.90 (m, 2H); 2.64 (s, 3H); 3.07 (t, 2H); 7.34 (m, 5H); 7.39 (d, 1H); 7.49 (s, 1H).

### 5.8 Example 8

### Preparation of 4-methyl-6-(1-methyl-1H-benzo [d]imidazol-2-yl)-2-propyl-1H-benzo[d]imidazole

The methyl ester, methyl 7-methyl-2-propyl-3H-benzo[d]imidazole-5-carboxylate (example 5) (1 mmol), N¹-methylbenzene-1,2-diamine (1 mmol) and polyphosphoric acid (5 mL) were mixed together and heated to 150° C. After 10 h the reaction mixture was diluted with water (20 mL) and the pH was adjusted to 8 by addition of concentrated ammonia, and the mixture was extracted with ethyl acetate. The combined extracts were dried, the solvent was evaporated, and the residue was purified by column chromatography gave 4-Methyl-6-(1-methyl-1H-benzo[d]imidazol-2-yl)-2-propyl-1H-benzo[d]imidazole(yield 45%).

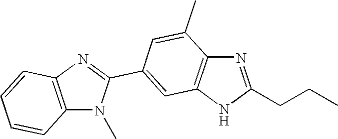

¹HNMR(CDCl₃, 400 MHz,): δ 0.95 (t,3H); 1.8 (m, 2H); 2.55 (s, 3H); 2.81 (t, 2H); 3.87 (s, 3H); 7.43 (m, 4H); 7.52 (s, 1H); 7.82 (m, 1H).

### 5.9 Example 9

### Preparation of 2-(4-cyclohexylbutyl)-1H-benzo[d]imidazole

The 5-cyclohexylpentanoic acid (1 mmol), phenylenediamine (1 mmol) and polyphosphoric acid (5 mL) was mixed together and heated to 150° C. After 10 h the reaction mixture was diluted with water (20 mL) and the pH was adjusted to 8 by addition of concentrated ammonia, and the mixture was extracted with ethyl acetate. The combined extracts were dried, the solvent was evaporated, and the residue was purified by column chromatography gave 2-(4-cyclohexylbutyl)-1H-benzo[d]imidazole (yield 65%).

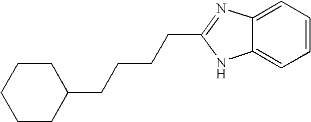

¹HNMR(CDCl₃, 400 MHz,): δ 0.82 (m, 2H); 1.15 (m, 6H); 1.36 (m, 2H); 1.62 (m, 5H); 1.83 (m, 2H); 2.92 (t, 2H); 7.21 (dd, 2H), 7.54 (dd, 2H).

### 5.10 Example 10

### N-alkylation of 4-methyl-6-(1-methyl-1H-benzo [d]imidazol-2-yl)-2-propyl-1H-benzo[d]-imidazole

To a 60% dispersion of NaH (48 mg, 1.2 mmol ) in anhydrous THF (10 mL) was added a solution of 4-methyl-6-(1-methyl-1H-benzo[d]imidazol-2-yl)-2-propyl-1H-benzo[d]-imidazole (1 mmol) in THF (10 mL) at 0° C. After stirring for 30 min at 0° C., the ethyl bromo acetate (1.2 mmol) was added, and stirring was continued until completion of the reaction. The reaction was quenched with saturated ammonium chloride and extracted with EtOAc. The combined organic layers were washed with brine, dried (MgSO₄) and purified by column chromatography to give ethyl 2-(4-methyl-6-(1-methyl-1H-benzo[d]imidazol-2-yl)-2-propyl-1H-benzo[d]-imidazol-1-yl)acetate (yield 92%).

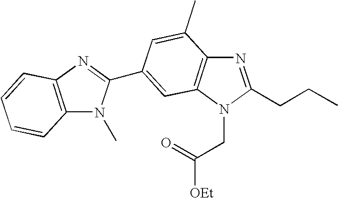

¹HNMR (400 MHz, CDCl₃): 1.05 (t, 3H); 1.21 (t, 3H); 1.85 (m, 2H); 2.70 (s, 3H); 2.87 (t, 2H); 3.83 (s, 3H); 4.21 (q, 2H); 4.87 (s, 2H); 7.46 (m, 4H); 7.52 (s, 1H); 7.80 (m, 1H).

### 5.11 Example 11

### Reduction of ethyl 2-(4-methyl-6-(1-methyl-1H-benzo[d]imidazol-2-yl)-2-propyl-1H-benzo[d]-imidazol-1-yl)acetate

To a solution of ethyl 2-(4-methyl-6-(1-methyl-1H-benzo[d]imidazol-2-yl)-2-propyl-1H-benzo[d]-imidazol-1-yl)acetate (1 mmol) in anhydrous THF (20 mL) lithium aluminium hydride (38 mg, 1 mmol) was added at 0° C. After 30 min, the reaction mixture was quenched with saturated sodium sulphate (2 mL) and stirring continued for an additional 30 min. The reaction mixture was filtered, and the filtrate was concentrated in vacuo to give a residue, 2-(4-methyl-6-(1-methyl-1H-benzo[d]imidazol-2-yl)-2-propyl-1H-benzo[d]imidazol-1-yl)ethanol, that was purified by column chromatography (yield 95%).

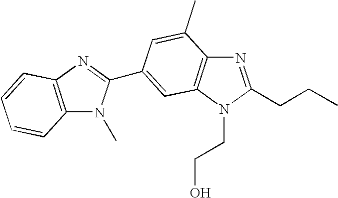

¹HNMR (CDCl₃, 400 MHz,): δ 1.02 (t, 3H); 1.81 (m, 2H); 2.68 (S, 3H); 2.81 (t, 2H); 3.48 (s, 3H); 3.78 (bs, 2H); 4.18 (bs, 2H); 7.32 (m, 5H); 7.82 (m, 1H).

### 5.12 Example 12

### Preparation of 2-(1-(2-bromoethyl)-4-methyl-2-propyl-1H-benzo[d]imidazol-6-yl)-1-methyl-1H-benzo d]imidazole

A 50 ml round bottom flask was charged with 2-(4-methyl-6-(1-methyl-1H-benzo[d]imidazol-2-yl)-2-propyl-1H-benzo[d]imidazol-1-yl)ethanol (1 mmol), CBr₄ (0.4 g, 1.2 mmol), and CH₂Cl₂ (20 mL) and cooled to 0° C. Over the course of 5 min, PPh₃ (0.32 g, 1.2 mmol) was added in portions. The reaction was allowed to reflux for 2 h. After completion of the reaction, the solvent was removed by rotary evaporation, and the crude reaction mixture was purified by column chromatography, yielding 1-(2-bromoethyl)-4-methyl-6-(1-methyl-1H-benzo[d]imidazol-2-yl)-2-propyl-1H-benzo[d]imidazole (yield 58%).

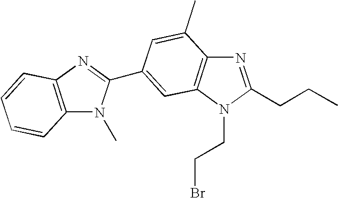

¹HNMR (CDCl₃, 400 MHz,): δ 1.06 (t, 3H); 1.89 (m, 2H); 2.70 (s, 3H); 2.90 (t, 2H) 3.62 (t, 2H); 3.80 (s, 3H); 4.96 (t, 2H); 7.29 (m, 4H); 7.55 (s, 1H); 7.80 (d, 1H).

### 5.13 Example 13

### Preparation of Pro-averysartan

To a solution of 4-methyl-6-(1-methyl-1H-benzo[d]imidazol-2-yl)-2-propyl-1H-benzo[d]imidazole, as prepared in Example 8, (1 mmol) in DMF (5 ml) was added anhydrous cesium carbonate (0.72 g, 2 mmol) and dimethyl (4′-bromomethyl)benzene-1,3-dioate as in example 2 (1.2 mmol) in DMF (5 ml) at 60° C. After 5 h, water was added, and the crude product was extracted with EtOAc, washed with brine, dried over MgSO4, and concentrated in vacuo. The residue, purified by column chromatography on silica gel gave proaverysartan (yield 35%).

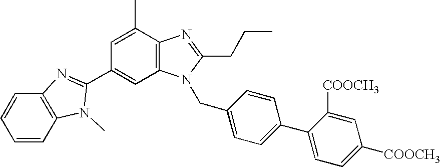

¹HNMR (CDCl₃, 400 MHz,): δ 1.04 (t, 3H); 1.85 (m, 2H); 2.76 (s, 3H); 2.91 (t, 2H); 3.60 (s, 3H); 3.77 (s, 3H), 3.92 (s, 3H); 5.43 (s, 2H); 7.09 (d, 2H); 7.27 (m, 6H); 7.43 (d, 2H); 7.77 (m, 1H); 8.13 (d, 1H); 8.46 (s, 1H).

### 5.14 Example 14

### Preparation of Averysartan

The ester, proaverysartan, (1.81 mmol) was dissolved in THF (10 mL) and water (2.5 mL) was added. To this mixture was added LiOH (0.062 g, 2.59 mmol) in water (1 mL), and the mixture was left overnight at RT. THF was removed by rotary evaporation, and conc HCl (0.25 mL) was added. The layers were separated, and the aq. layer was extracted with ethylacetate (25 mL). The organic layer was washed with water and dried over MgSO₄ and concentrated to give the acid, averysartan (yield 85%).

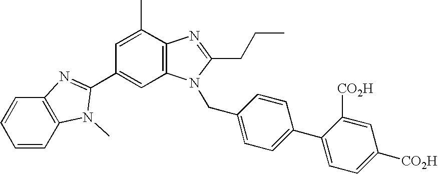

¹HNMR (MeOH-d₄,400 MHz): δ 1.06 (t, 3H); 1.82 (m, 2H); 2.73 (s, 3H); 3.06 (t, 2H); 3.82 (s, 3H); 5.64 (s, 2H); 7.20 (d, 2H); 7.43 (m, 6H); 7.60 (m, 2H); 7.74 (d, 1H); 8.10 (d, 1H); 8.35 (s, 1H).

### 5.15 Example 15

### Preparation of Methyl 5-((2,4-dioxothiazolidin-5-yl)methyl)-2-(2-(4-methyl-6-(1-methyl-1H-benzo[d]imidazol-2-yl)-2-propyl-1H-benzo[d]imidazol-1-yl)ethoxy)benzoate

To a solution of methyl 5-((2,4-dioxothiazolidin-5-yl)methyl)-2-hydroxybenzoate, as prepared in Example 4, (1 mmol) in DMF (5 ml) was added anhydrous cesium carbonate (0.72 g, 2 mmol) and 2-(1-(2-bromoethyl)-4-methyl-2-propyl-1H-benzo[d]imidazol-6-yl)-1-methyl-1H-benzo[d]imidazole as prepared in example 12 (1.2 mmol) in DMF (5 ml) at 60° C. After 5 h, water was added, and the crude product was extracted with EtOAc, washed with brine, dried over MgSO4, and concentrated in vacuo. The residue, purified by column chromatography on silica gel gave methyl 5-((2,4-dioxothiazolidin-5-yl)methyl)-2-(2-(4-methyl-6-(1-methyl-1H-benzo[d]imidazol-2-yl)-2-propyl-1H-benzo[d]imidazol-1-yl)ethoxy)benzoate (yield 55%).

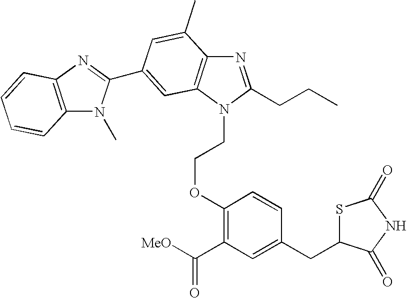

¹HNMR (CDCl₃, 400 MHz,): δ 1.14 (t, 3H); 1.98 (m, 2H); 2.74 (s, 3H); 2.80 (dd, 1H); 2.94 (m, 2H); 3.33 (dd, 1H); 3.86 (s, 3H); 3.95 (s, 3H); 3.98 (m, 2H); 4.35 (m, 2H); 4.56 (m, H); 7.51 (s, 1H); 7.61 (d, 2H); 7.83 (d, 1H).

### 5.16 Example 16

### N-alkylation of 6-bromo-4-methyl-2-propyl-1H-benzo[d]imidazole

To a 60% dispersion of NaH (48 mg, 1.2 mmol ) in anhydrous THF (10 mL) was added a solution of 6-bromo-4-methyl-2-propyl-1H-benzo[d]imidazole (1 mmol) in THF (10 mL) at 0° C. After stirring for 30 min at 0° C., the ethyl bromo acetate (1.2 mmol) was added, and stirring was continued until completion of the reaction. The reaction was quenched with saturated ammonium chloride and extracted with EtOAc. The combined organic layers were washed with brine, dried (MgSO₄) and purified by column chromatography to give ethyl 2-(6-bromo-4-methyl-2-propyl-1H-benzo[d]imidazol-1-yl)acetate (yield 95%).

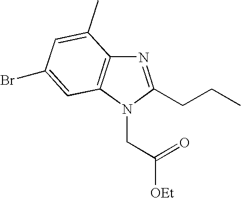

¹HNMR(CDCl₃, 400 MHz,): δ 1.04 (t, 3H); 1.26 (t, 3H); 1.85 (m, 2H); 2.61 (s, 3H); 2.80 (t, 2H); 4.23 (s, 2H); 4.74 (s, 2H); 7.17 (bs, 2H)

### 5.17 Example 17

### Reduction of ethyl 2-(6-bromo-4-methyl-2-propyl-1H-benzo[d]imidazol-1-yl)acetate

To a solution of ethyl 2-(6-bromo-4-methyl-2-propyl-1H-benzo[d]imidazol-1-yl)acetate (1 mmol) in anhydrous THF (20 mL) lithium aluminium hydride (38 mg, 1 mmol) was added at 0° C. After 30 min, the reaction mixture was quenched with saturated sodium sulphate (2 mL) and stirring continued for an additional 30 min. The reaction mixture was filtered, and the filtrate was concentrated in vacuo to give a residue, 2-(6-Bromo-4-methyl-2-propyl-1H-benzo[d]imidazol-1-yl)ethanol, that was purified by column chromatography(yield 95%).

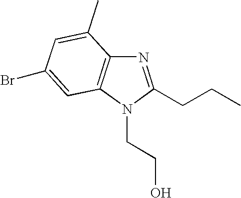

¹HNMR (CDCl₃, 400 MHz,): δ 0.98 (t, 3H); 1.72 (m, 2H); 2.39 (s, 3H); 2.83 (t, 2H); 4.01 (t, 2H); 4.15 (t, 2H); 6.99 (s, 1H); 7.22 (s, 1H).

### 5.18 Example 18

### Preparation of 2-(6-bromo-4-methyl-2-propyl-1H-benzo[d]imidazol-1-yl)ethyl methanesulfonate

To a solution of 2-(6-Bromo-4-methyl-2-propyl-1H-benzo[d]imidazol-1-yl)ethanol (10 mmol) in CH₂Cl₂ (15 ml) was added NEt₃ (2.0 ml, 14.41 mmol) and MsCl (1.1 ml, 14.41 mmol) at 0° C. After stirring for 2 h at room temperature, the reaction mixture was washed with H₂O, dried over MgSO₄, and concentrated in vacuo. The residue was purified by column chromatography on silica gel to give 2-(6-bromo-4-methyl-2-propyl-1H-benzo[d]imidazol-1-yl)ethyl methanesulfonate as colorless oil (yield 83%).

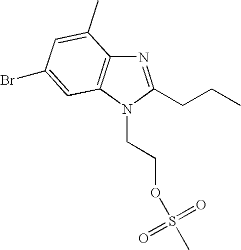

¹HNMR (CDCl₃, 400 MHz,): δ 1.08 (t, 3H); 1.88 (m, 2H); 2.61 (s, 3H); 2.81 (s, 3H); 2.87 (t, 2H); 4.41 (t, 2H); 4.48 (t, 2H); 7.18 (s, 1H); 7.30 (s, 1H).

### 5.19 Example 19

### Methyl 2-(2-(6-bromo-4-methyl-2-propyl-1H-benzo[d]imidazol-1-yl)ethoxy)-5-((2,4-dioxothiazolidin-5-yl)methyl)benzoate

To a solution of methyl 5-((2,4-dioxothiazolidin-5-yl)methyl)-2-hydroxybenzoate, as prepared in Example 4, (1 mmol) in DMF (5 ml) was added anhydrous cesium carbonate (0.72 g, 2 mmol) and 2-(6-bromo-4-methyl-2-propyl-1H-benzo[d]imidazol-1-yl)ethyl methanesulfonate as prepared in example 18 (1.2 mmol) in DMF (5 ml) at 60° C. After 5 h, water was added, and the crude product was extracted with EtOAc, washed with brine, dried over MgSO4, and concentrated in vacuo. The residue, purified by column chromatography on silica gel gave methyl 2-(2-(6-bromo-4-methyl-2-propyl-1H-benzo[d]imidazol-1-yl)ethoxy)-5-((2,4-dioxothiazolidin-5-yl)methyl)benzoate (yield 45%).

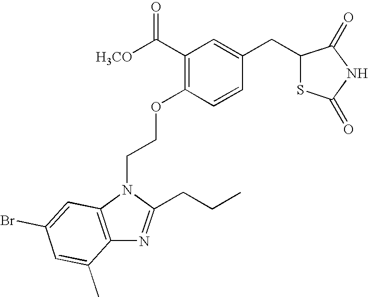

¹HNMR (CDCl₃, 400 MHz,): δ 0.99 (t, 3H); 1.80 (m, 2H), 2.45 (s, 3H); 2.87 (t, 2H); 3.06 (dd, 1H); 3.26 (dd, 1H); 3.68 (s, 3H); 4.31 (bs, 2H); 4.55 (bs, 2H); 4.85 (dd, 1H); 7.05 (d, 2H); 7.09 (s, 1H); 7.33 (d, 1H); 7.44 (s, 1H); 7.64 (s, 1H).

### 5.20 Example 20

### Preparation of 2-(2-(6-Bromo-4-methyl-2-propyl-1H-benzo[d]imidazol-1-yl)ethoxy)-5-((2,4-dioxothiazolidin-5-yl)methyl)benzoic acid

The ester, methyl 2-(2-(6-bromo-4-methyl-2-propyl-1H-benzo[d]imidazol-1-yl)ethoxy)-5-((2,4-dioxothiazolidin-5-yl)methyl)benzoate as prepared in example 19, (1.81 mmol) was dissolved in THF (10 mL) and water (2.5 mL) was added. To this mixture was added LiOH (0.062 g, 2.59 mmol) in water (1 mL), and the mixture was left overnight at RT. THF was removed by rotary evaporation, and conc HCl (0.25 mL) was added. The layers were separated, and the aq. layer was extracted with ethylacetate (25 mL). The organic layer was washed with water and dried over MgSO₄ and concentrated to give the acid, 2-(2-(6-Bromo-4-methyl-2-propyl-1H-benzo[d]imidazol-1-yl)ethoxy)-5-((2,4-dioxothiazolidin-5-yl)methyl)benzoic acid (yield 89%).

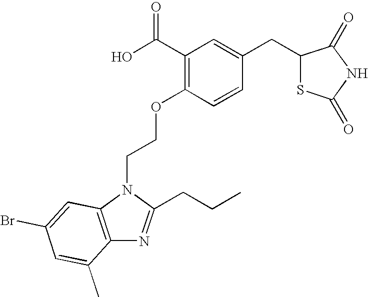

¹HNMR (MeOH-d₄, 400 MHz,): δ 1.10 (t, 3H); 1.78 (m, 2H), 2.43 (s, 3H); 2.93 (t, 2H); 3.16 (dd, 1H); 3.36 (dd, 1H); 4.43 (bs, 2H); 4.65 (bs, 2H); 4.89 (dd, 1H); 7.09 (d, 2H); 7.30 (s, 1H); 7.32 (d, 1H); 7.48 (s, 1H); 7.76 (s, 1H).

### 5.21 Example 21

### N-alkylation of 4-methyl-6-phenyl-2-propyl-1H-benzo[d]imidazole

To a 60% dispersion of NaH (48 mg, 1.2 mmol ) in anhydrous THF (10 mL) was added a solution of 4-methyl-6-phenyl-2-propyl-1H-benzo[d]imidazole (1 mmol) in THF (10 mL) at 0° C. After stirring for 30 min at 0° C., the ethyl bromo acetate (1.2 mmol) was added, and stirring was continued until completion of the reaction. The reaction was quenched with saturated ammonium chloride and extracted with EtOAc. The combined organic layers were washed with brine, dried (MgSO₄) and purified by column chromatography to give ethyl 2-(4-methyl-6-phenyl-2-propyl-1H-benzo[d]imidazol-1-yl)acetate (yield 90%).

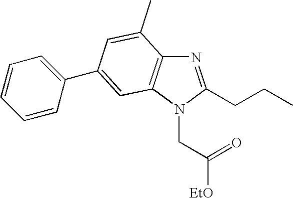

¹HNMR(CDCl₃, 400 MHz,): δ 1.09 (t, 3H); 1.29 (t, 3H); 1.91 (m, 2H); 2.74 (s, 3H); 2.89 (t, 2H); 4.25 (q, 2H); 4.87 (s, 2H), 7.23 (s, 1H); 7.33 (t, 2H); 7.46 (t, 2H); 7.63 (d, 2H)

### 5.22 Example 22

### Reduction of ethyl 2-(4-methyl-6-phenyl-2-propyl-1H-benzo[d]imidazol-1-yl)acetate

To a solution of ethyl 2-(4-methyl-6-phenyl-2-propyl-1H-benzo[d]imidazol-1-yl)acetate (1 mmol) in anhydrous THF (20 mL) lithium aluminium hydride (38 mg, 1 mmol) was added at 0° C. After 30 min, the reaction mixture was quenched with saturated sodium sulphate (2 mL) and stirring continued for an additional 30 min. The reaction mixture was filtered, and the filtrate was concentrated in vacuo to give a residue, 2-(4-methyl-6-phenyl-2-propyl-1H-benzo[d]imidazol-1-yl)ethanol, that was purified by column chromatograph (yield 97%).

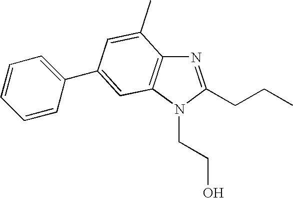

¹HNMR (CDCl₃, 400 MHz,): δ 0.97 (t, 3H); 1.71 (m, 2H); 2.39 (s, 3H); 2.87 (t, 2H); 4.07 (bs, 2H); 4.24 (bs, 2H); 7.05 (s, 1H); 7.26 (s, 1H); 7.35 (t, 1H); 7.44 (t, 2H); 7.59 (d, 2H).

### 5.23 Example 23

### Preparation of Bromide

A 50 ml round bottom flask was charged with 2-(4-Methyl-6-phenyl-2-propyl-1H-benzo[d]imidazol-1-yl)ethanol (1 mmol), CBr₄ (0.4 g, 1.2 mmol), and CH₂Cl₂ (20 mL) and cooled to 0° C. Over the course of 5 min, PPh₃ (0.32 g, 1.2 mmol) was added in portions. The reaction was allowed to reflux for 2 h. After completion of the reaction, the solvent was removed by rotary evaporation, and the crude reaction mixture was purified by column chromatography, yielding 1-(2-bromoethyl)-4-methyl-6-phenyl-2-propyl-1H-benzo[d]imidazole (yield 75%).

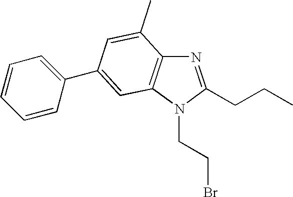

¹HNMR (CDCl₃, 400 MHz,): δ 1.08 (t, 3H; 1.89 (m, 2H); 2.70 (s, 3H); 3.01 (t, 2H); 3.85 (t, 2H); 4.51 (t, 2H); 7.34 (m, 3H); 7.43 (t, 2H); 7.61 (d, 2H)

### 5.24 Example 24

### Preparation of 2-(4-methyl-6-phenyl-2-propyl-1H-benzo d]imidazol-1-yl)ethyl methanesulfonate

To a solution of 2-(4-methyl-6-phenyl-2-propyl-1H-benzo[d]imidazol-1-yl)ethanol (10 mmol) in CH₂Cl₂ (15 ml) was added NEt₃ (2.0 ml, 14.41 mmol) and MsCl (1.1 ml, 14.41 mmol) at 0° C. After stirring for 2 h at room temperature, the reaction mixture was washed with H₂O, dried over MgSO₄, and concentrated in vacuo. The residue was purified by column chromatography on silica gel to give 2-(4-methyl-6-phenyl-2-propyl-1H-benzo[d]imidazol-1-yl)ethyl methanesulfonate as colorless oil (yield 98%).

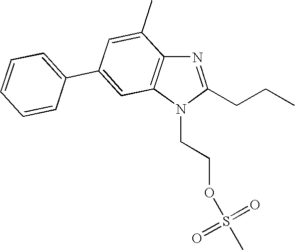

¹HNMR (CDCl₃, 400 MHz,): δ 1.10 (t, 3H); 1.93 (m, 2H); 2.73 (s, 3H); 2.77 (s, 3H); 2.95 (t, 2H); 4.54 (bs, 4H); 7.35 (m, 3H); 7.46 (t, 2H); 7.65 (d, 2H).

### 5.25 Example 25

### Preparation of Methyl 5-((2,4-dioxothiazolidin-5-yl)methyl)-2-(2-(4-methyl-6-phenyl-2-propyl-1H-benzo[d]imidazol-1-yl)ethoxy)benzoate

To a solution of methyl 5-((2,4-dioxothiazolidin-5-yl)methyl)-2-hydroxybenzoate, as prepared in Example 4, (1 mmol) in DMF (5 ml) was added anhydrous cesium carbonate (0.72 g, 2 mmol) and 2-(4-methyl-6-phenyl-2-propyl-1H-benzo[d]imidazol-1-yl)ethyl methanesulfonate as prepared in example in 24 (1.2 mmol) in DMF (5 ml) at 60° C. After 5 h, water was added, and the crude product was extracted with EtOAc, washed with brine, dried over MgSO4, and concentrated in vacuo. The residue, purified by column chromatography on silica gel gave methyl 5-((2,4-dioxothiazolidin-5-yl)methyl)-2-(2-(4-methyl-6-phenyl-2-propyl-1H-benzo[d]imidazol-1-yl)ethoxy) benzoate (yield 65%).

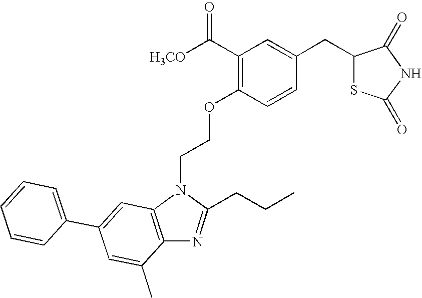

¹HNMR (CDCl₃, 400 MHz,): δ 1.02 (t, 3H); 1.82 (m, 2H); 2.54 (s, 3H); 2.92 (t, 2H); 3.07 (dd, 1H); 3.27 (dd, 1H); 3.62 (s, 3H); 4.38 (bs, 2H); 4.62 (bs, 2H); 4.85 (dd, 1H); 7.08 (d, 1H); 7.26 (s, 1H); 7.31 (dd, 2H); 7.43 (m, 3H); 7.64 (s, 1H); 7.70 (d, 2H).

### 5.26 Example 26

### N-alkylation of 2-(4-cyclohexylbutyl)-1H-benzo[d]imidazole

To a 60% dispersion of NaH (48 mg, 1.2 mmol ) in anhydrous THF (10 mL) was added a solution of 2-(4-cyclohexylbutyl)-1H-benzo[d]imidazole as prepared in example 9 (1 mmol) in THF (10 mL) at 0° C. After stirring for 30 min at 0° C., the ethyl bromo acetate (1.2 mmol) was added, and stirring was continued until completion of the reaction. The reaction was quenched with saturated ammonium chloride and extracted with EtOAc. The combined organic layers were washed with brine, dried (MgSO₄) and purified by column chromatography to give ethyl 2-(2-(4-cyclohexylbutyl)-1H-benzo[d]imidazol-1-yl)acetate (yield 96%).

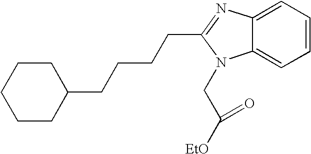

¹HNMR(CDCl₃, 400 MHz,): δ 0.87 (m, 2H); 1.21 (m, 9H); 1.46 (m, 2H); 1.72 (m, 5H); 1.90 (m, 2H); 2.83 (t, 2H); 4.25 (q, 2H); 4.83 (s, 2H); 7.25 (m, 3H); 7.45 (d, 1H).

### 5.27 Example 27

### Reduction of ethyl 2-(2-(4-cyclohexylbutyl)-1H-benzo[d]imidazol-1-yl)acetate

To a solution of ethyl 2-(2-(4-cyclohexylbutyl)-1H-benzo[d]imidazol-1-yl)acetate (1 mmol) in anhydrous THF (20 mL) lithium aluminium hydride (38 mg, 1 mmol) was added at 0° C. After 30 min, the reaction mixture was quenched with saturated sodium sulphate (2 mL) and stirring continued for an additional 30 min. The reaction mixture was filtered, and the filtrate was concentrated in vacuo to give a residue, 2-(2-(4-Cyclohexylbutyl)-1H-benzo[d]imidazol-1-yl)ethanol, that was purified by column chromatography (yield 90%).

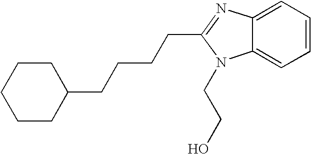

¹HNMR (CDCl₃, 400 MHz,): δ 0.86 (m, 2H); 1.16 (m, 5H); 1.26 (m, 3H), 1.55 (m, 2H); 1.68 (m, 5H); 2.54 (t, 2H); 4.04 (t, 2H); 4.19 (t, 2H); 7.09 (t, 1H); 7.14 (t, 1H); 7.25 (d, 1H); 7.40 (d, 1H).

### 5.28 Example 28

### Preparation of 2-(2-(4-Cyclohexylbutyl)-1H-benzo [d]imidazol-1-yl)ethyl methanesulfonate

To a solution of 2-(2-(4-Cyclohexylbutyl)-1H-benzo[d]imidazol-1-yl)ethanol (10 mmol) in CH₂Cl₂ (15 ml) was added NEt₃ (2.0 ml, 14.41 mmol) and MsCl (1.1 ml, 14.41 mmol) at 0° C. After stirring for 2 h at room temperature, the reaction mixture was washed with H₂O, dried over MgSO₄, and concentrated in vacuo. The residue was purified by column chromatography on silica gel to give 2-(2-(4-Cyclohexylbutyl)-1H-benzo[d]imidazol-1-yl)ethyl methanesulfonate as colorless oil (yield 98%).

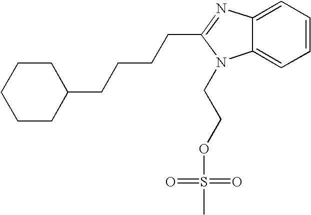

¹HNMR (CDCl₃, 400 MHz,): δ 0.81 (m, 2H); 1.13 (m, 5H); 1.40 (m, 2H); 1.62 (m, 5H); 1.82 (m, 2H); 2.60 (s, 3H); 2.80 (t, 2H); 4.36 (bs, 4H); 7.18 (m, 2H); 7.23 (d, 1H); 7.66 (d, 1H).

### 5.29 Example 29

### Preparation of Methyl 2-(2-(2-(4-cyclohexylbutyl)-1H-benzo[d]imidazol-1-yl)ethoxy)-5-((2,4-dioxothiazolidin-5-yl)methyl)benzoate

To a solution of methyl 5-((2,4-dioxothiazolidin-5-yl)methyl)-2-hydroxybenzoate, as prepared in Example 4, (1 mmol) in DMF (5 ml) was added anhydrous cesium carbonate (0.72 g, 2 mmol) and 2-(2-(4-Cyclohexylbutyl)-1H-benzo[d]imidazol-1-yl)ethyl methanesulfonate as prepared in Example 28 (1.2 mmol) in DMF (5 ml) at 60° C. After 5 h, water was added, and the crude product was extracted with EtOAc, washed with brine, dried over MgSO4, and concentrated in vacuo. The residue, purified by column chromatography on silica gel gave methyl 2-(2-(2-(4-cyclohexylbutyl)-1H-benzo[d]imidazol-1-yl)ethoxy)-5-((2,4-dioxothiazolidin-5-yl)methyl)benzoate (yield 43%).

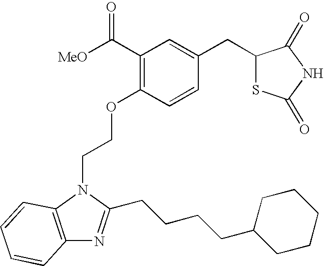

¹HNMR (CDCl₃, 400 MHz,): δ 0.87 (m, 2H); 1.21 (m, 5H); 1.46 (m, 2H); 1.67 (m, 5H); 1.83 (m, 2H); 3.0 (t, 2H); 3.07 (dd, 1H); 3.42 (dd, 1H); 3.82 (s, 3H); 4.32 (t, 2H); 4.43 (dd, 1H); 4.58 (t, 2H); 6.72 (d, 1H); 7.23 (m, 3H); 7.38 (m, 1H); 7.63 (bs, 1H); 7.77 (dd, 1H).

### 5.30 Example 30

### Determination of PPARγ Activity

PPARγ activity was determined by transactivation assays in CV-1 cells (CCL-70 line from American Type Culture Collection (ATCC), Bethesda, Md.) transfected using the GenePorter transfection reagent (Gene Therapy Systems, San Diego, Calif.) to deliver 200 ng of a PPARγ expression plasmid and 1 μg of a luciferase reporter plasmid and 400 ng pCMVSport b-gal (Gibco, Grand Island, N.J.) as an internal control. 24 hr post-transfection, cells were treated with varying concentrations of the test compounds of this invention, and incubated for an additional 24 hr. Rosiglitazone and pioglitazone, a known PPARγ full agonist, and telmisartan, a known ARB and PPARγ partial agonist (Benson et al. Hypertension (2004) in press) are used as controls. Cell extracts were assayed for luciferase and β-galactosidase activity using Promega (Madison, Wis.) assay systems. All treatments were performed in triplicate, and normalized for β-galactosidase activity. Agonist concentrations yielding half maximal activation (EC50 values) were calculated using GraphPad Prism version 3.03 (GraphPad Software, Inc., San Diego, Calif.).

Telmisartan significantly activated PPARγ (5-8 fold) when tested at concentrations (1-5 mM) that can be achieved in plasma with conventional oral dosing (Stangier J, et al. J Int Med Res (2000) 28:149-67). Telmisartan functioned as a moderately potent (EC50=4.5 mM), PPARγ agonist, activating the receptor to 25%-30% of the maximum level of activity achieved by the full agonists pioglitazone and rosiglitazone. Irbesartan activated PPARγ (2-3 fold activation) when tested at 10 mM. None of the other ARBs tested caused any significant activation of PPAR even when tested at higher concentrations (more than 10 mM). These experiments demonstrate that two known ARBs, telmisartan and irbesartan, are also activators of PPARγ. Because PPARγ activators can be used to treat and prevent type 2 diabetes, the metabolic syndrome, and other clinical disorders responsive to treatment with PPAR activators, these experiments demonstrate the utility of telmisartan and irbesartan for the prevention and treatment of type 2 diabetes, the metabolic syndrome, and other disorders known to be responsive to treatment with PPAR activators.

| | | PPAR-γ |
|---|---|---|
| | Mol. Form | Fold |
| Structure | (M. Wt.) | activation |
| 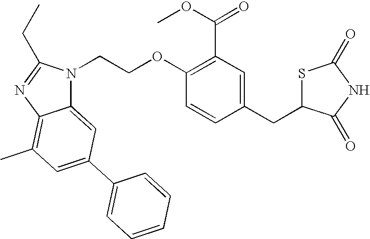 | C₃₁H₃₁N₃O₅S (557.66) | 7.4x @ 10 μM 6.1x @ 1 μM 1.2x @ 0.1 uM |
| 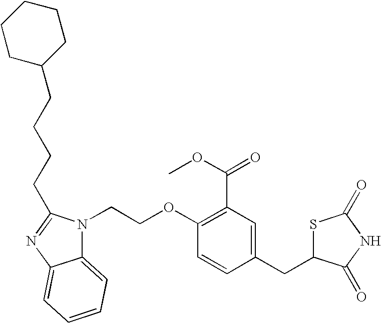 | C₃₁H₃₇N₃O₅S (563.71) | 34x @ 10 uM 24x @ 1 uM 2x @ .1 uM 1x @ .01 uM |
| 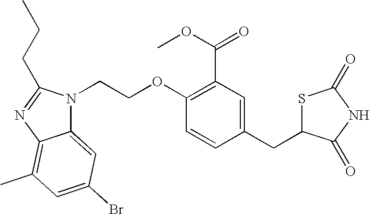 | C₂₅H₂₆BrN₃O₅S (560.46) | 41x @ 10 uM 32x @ 1 uM 3.1x @ .1 uM 1x @ .01 uM |
| 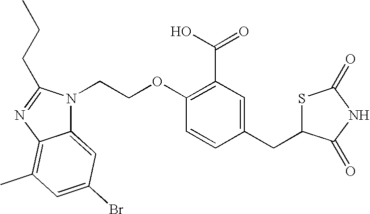 | C₂₄H₂₄BrN₃O₅S (546.43) | 15.5x @ 10 uM 10x @ 1 uM 3.8x @ .1 uM 1.7x @ .01 uM |
| 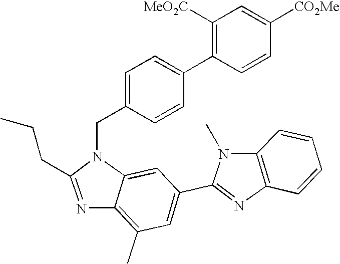 | C₃₆H₃₄N₄O₄(586.68) | 11x @ 10 uM 9x @ 5 uM 5x @ 1 uM 1x @ 0.1 uM |
| 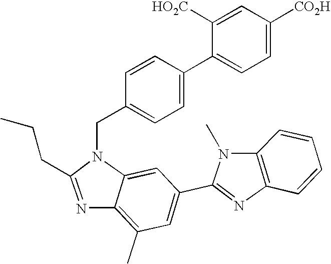 | C₃₄H₃₀N₄O₄(558.63) | No activity |

### 5.31 Example 31

### Angiotensin II Receptor Binding Assay

Chinese Hamster Ovary (CHO) cells stably transfected and expressing the human angiotensin II receptor were used for this assay. Cells were cultured to confluence in DMEM supplemented with 10% fetal bovine serum, 100 mM proline, 1 mg/ml geneticin and antibiotics in wells of a 24 well dish. At the time of assay cells were washed with phosphate buffered saline (PBS) and preincubated for 15 minutes with test compounds (10⁻¹¹-10⁻⁶ M) to allow binding to the receptor. Test compounds were dissolved in DMEM, 0.2% BSA and included potential and verified receptor agonists and antagonists. Following the 15 min preincubation period, cells were incubated with I¹²⁵-angiotensin for 60 minutes. Following the 60 minute incubation, cells were washed five times with PBS, then dissolved in 1 N NaOH for 15 minutes at 37° C. Aliquots of the solubilized cells were quantitated for radioactive ¹²⁵ I-angiotensin using a gamma counter.

### 5.32 Example 32

### Measurement of in Vivo Insulin Sensitivity Activity

Activity of in vivo efficacy of a test compound as an insulin-sensitizing agent is measured as the insulin and glucose-lowering activity of said test compound in the dietary model of insulin resistance. Male Sprague-Dawley rats are placed on a high fat, high carbohydrate diet (Teklad Diet, TD03203 containing 60% fructose, 10% lard, and 0.06% magnesium) at 6 weeks of age. Two days after starting the diet, the rats were randomized into 3 different groups (n=10 rats per group): Group 1, is treated with the test compound (10 mg/kg body weight per day); Group 2, is the positive control group, telmisartan (10 mg/kg body weight per day); Group 3, is the negative control group (no drug). The compounds were administered by dissolving the commercially available medications in the drinking water at an initial concentration of 40 mg/liter. Food and fluid intakes are measured each day and a pair-feeding protocol is utilized to insure equivalent food intakes among the 3 groups. After 5 weeks, serum levels of glucose, insulin, and triglycerides are obtained in the semi-fasting state (the night before blood draw, animals are given a restricted amount of chow equivalent to 3 grams/100 gram body weight at 5 pm and blood and drawn the following morning from the tail vein in the unanesthetized state). The protocol is continued for an additional 9 weeks at which time glucose tolerance testing (OGTT) is performed in conscious animals in the semi-fasted state by sampling blood for glucose and insulin measurements after oral administration of a glucose load (100 mg/100 gram body weight). Serum levels of glucose and triglycerides are measured by spectrophotometric methods and insulin levels were measured by radioimmunoassay (Linco, St. Louis, Mo.). Statistical analysis of the data are performed by Student's t test and ANOVA followed by Dunnett's multiple comparison test or the Student-Newman-Keuls test for comparisons across multiple groups. Statistical significance is defined as P<0.05. The insulin-sensitizing activity of the test compounds are calculated as follows:

Insulin-sensitivity activity (%)={[(PI in C)−(PI in T)]/[PI in C]}×100 where “PI in C” is plasma insulin in control rats and “PI in T” is plasma insulin in rats treated with test compounds.

### 5.33 Example 33

### A Clinical Trial Using a Dual PPARγ Activator to Treat Type 2 Diabetes Without Causing Fluid Retention, Edema, or Heart Failure

For the purpose of illustration telmisartan (Micardis®), an angiotensin II type 1 receptor blocker (ARB) which has been shown to activate PPARγ (Benson et al. Hypertension (2004) in press), is used to demonstrate similar clinically beneficial effects anticipated by compounds of this invention which are also dual PPARγ partial agonists and antagonize the angiotensin II type 1 receptor. A 49 year old female with hypertension, hypertriglyceridemia, and type 2 diabetes was selected for therapy. Before administration of telmisartan (Micardis®), the patient had a blood pressure of 147/92 mmHg, fasting serum glucose of 183 mg/dl, a fasting serum triglyceride level of 264 mg/dl, and an HDL cholesterol level of 48 mg/dl. The patient is taking another medication for type 2 diabetes but the dose of this medication is held constant throughout the trial. The patient is given telmisartan at an oral dose of 80 mg/day. After three weeks of telmisartan therapy, the blood pressure is reduced to 143/91 mmHg with little or no improvement in fasting glucose (188 mg/dl), triglyceride (281 mg/dl), or HDL cholesterol levels (50 mg/dl). The oral dose of telmisartan (Micardis®) is then increased to 160 mg/day. After seven weeks of telmisartan (Micardis®) therapy at 160 mg/day, the patient's blood pressure is reduced to 131/81 mmHg and there is a significant improvement in the diabetes with the glucose level reduced to 145 mg/dl, the triglyceride level reduced to 178 mg/dl, and the HDL cholesterol increased to 60 mg/dl. Clinical examination reveals no evidence of any increase in fluid retention, peripheral edema, pulmonary edema, or congestive heart failure. The telmisartan (Micardis®) therapy is continued according to the judgment of the clinician in order to maintain the improved control of the patient's blood pressure and her type 2 diabetes.

### 5.34 Example 34

### A Clinical Trial Using a Dual ARB/PPARγ Activator to Treat the Metabolic Syndrome Without Causing Fluid Retention, Edema, or Heart Failure

For the purpose of illustration telmisartan (Micardis®), an angiotensin II type 1 receptor blocker (ARB) which has been shown to activate PPARγ (Benson et al. Hypertension (2004) in press), is used to demonstrate similar clinically beneficial effects anticipated by compounds of this invention which are also dual PPARγ partial agonists and antagonize the angiotensin II type 1 receptor. An obese 52-year-old man (BMI: 34.4 kg/m2, waist circumference: 51 inches) with untreated hypertension, pre-diabetes and the metabolic syndrome was given telmisartan (Micardis®), 80 mg/day, for treatment of high blood pressure. Before administration of telmisartan, the patient had a blood pressure of 160/79 mmHg, fasting serum glucose of 123 mg/dl, fasting insulin level of 30 μU/ml, fasting triglycerides of 136 mg/dl, and waist girth of 120 cm. The patient has the metabolic syndrome as defined by the National Cholesterol Education Program (NCEP). The metabolic syndrome is associated with a 5-9 fold increase in the risk for developing type 2 diabetes and a 2-3 fold increase risk in cardiovascular mortality. In addition to normalization of his blood pressure, his blood glucose, insulin and triglyceride concentrations also progressively decreased, with an improvement in insulin resistance based on the homeostasis model assessment of insulin resistance (HOMA-IR) score (Table 1). His blood pressure normalized and remained normal throughout the observation period. At week 10, he was switched to a therapeutically equivalent dose of valsartan (Diovan®), an ARB that does not activate PPARγ (Benson et al. Hypertension (2004) in press), at 160 mg/day. After 5 weeks on valsartan (week 15), his glucose, insulin, triglyceride levels, and HOMA-IR score increased, consistent with a reversal in the improved insulin resistance obtained with telmisartan (Table 1). Valsartan was discontinued and he was switched back to telmisartan. Over the following 4 weeks, his insulin resistance and triglycerides again improved (week 19). Clinical examination reveals no evidence of any increase in fluid retention, peripheral edema, pulmonary edema, or congestive heart failure.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the appended claims.

All publications and patents cited herein are incorporated by reference in their entirety

1. A compound of the structure: 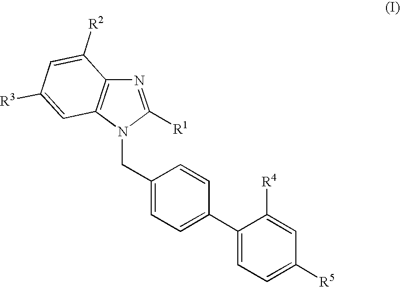
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
R⁴ and R⁵ independently are hydrogen, cyanate, or a negatively charged group.

2. The compound of claim 1, wherein:
R¹ is hydrogen, halogen, methoxy, hydroxyl, methyl, ethyl, or NH₂;
R² is lower alkyl;
R³ is phenyl, a fused 2-membered heterocyclic radical or 5- or 6-membered heterocyclic ring;
R⁴ is hydrogen or cyanate; and
R⁵ is a negatively charged group.

3. The compound of claim 2, wherein
R¹ is hydrogen or methyl;
R² is lower alkyl;
R³ is benzimidazole;
R⁴ is a hydrogen; and
R⁵ is alkoxycarbonyl or carboxyl.

4. The compound of claim 3, wherein R³ is: 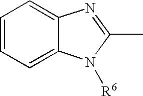
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

5. The compound of claim 1, wherein:
R¹ is alkyl of 3 or more carbons;
R² is hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
R³ is aryl, 5- or 6-membered heterocyclic ring, or a fused 2-, 3-, or 4-membered heterocyclic radical;
R⁴ is a negatively charged group; and
R⁵ is a negatively charged group.

6. The compound of claim 5, wherein
R¹ is propyl or butyl;
R² is lower alkyl;
R³ is phenyl, a fused 2-membered heterocyclic radical or 5- or 6-membered heterocyclic ring;
R⁴ is cyanate, 2-tetrazolyl, carboxyl, alkoxycarbonyl, carbamoyl, sulfonamido, or alkylsulfonamido; and
R⁵ is cyanate, 2-tetrazolyl, carboxyl, alkoxycarbonyl, carbamoyl, sulfonamido, or alkylsulfonamido.

7. The compound of claim 6, wherein
R¹ is propyl or butyl;
R² is lower alkyl;
R³ is benzimidazole;
R⁴ is lower alkoxycarbonyl or carboxyl; and
R⁵ is lower alkoxycarbonyl or carboxyl.

8. The compound of claim 7, wherein R³ is: 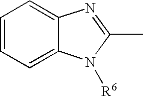
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

9. The compound of claim 8, wherein R⁶ is hydrogen or lower alkyl.

10. The compound of claim 9, wherein the compound has the structure:

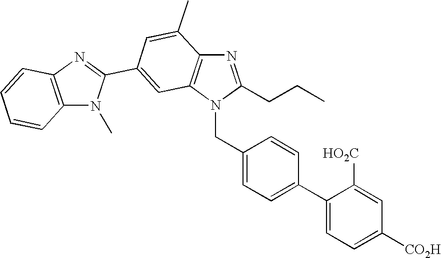

11. The compound of claim 9, wherein the compound has the structure:

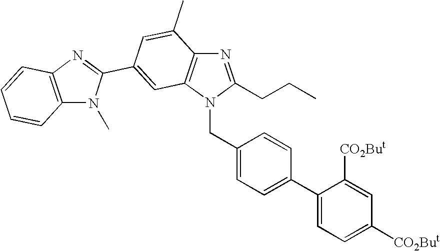

12. The compound of claim 1, wherein R⁴ and R⁵ independently are hydrogen or 2-tetrazolyl.

13. The compound of claim 1, wherein the compound at least partially activates a peroxisome proliferator-activated receptor.

14. The compound. of claim 1, wherein the compound at least partially activates PPARγ.

15. The compound of claim 14, wherein the compound does not inhibit activity of the AT1 receptor.

16. The compound of claim 14, wherein the compound at least partically inhibits activity of the AT1 receptor.

17. A compound of the structure: 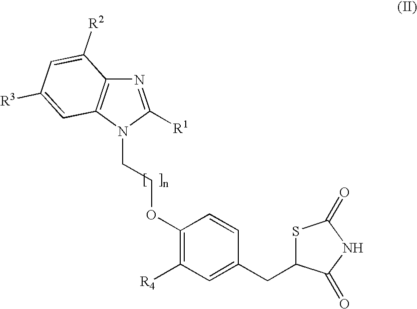
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
n is 0 to 2; and
R⁴ is hydrogen, cyanate, 2-tetrazolyl, carboxyl, alkoxycarbonyl, amido, or sulfonamido.

18. The compound of claim 17, wherein
R¹ is hydrogen, lower alkyl or cyclolower alkylalkyl;
R² is hydrogen, lower alkyl, halogen, hydroxyl or NH₂;
R³ is phenyl, halogen, hydrogen, amino, alkoxy, hydroxyl, 5- or 6-membered heterocyclic ring, or a fused 2-4-membered heterocyclic radical; and
R⁴ is hydrogen, cyanate, 2-tetrazolyl, carboxyl, alkoxycarbonyl, amido, or sulfonamido.

19. The compound of claim 18, wherein
n is 1;
R¹ is hydrogen, methyl, ethyl or 4-cyclohexylbutyl;
R² is hydrogen, or methyl;
R³ is phenyl, halogen, or benzimidazole; and
R⁴ is loweralkoxycarbonyl or carboxyl.

20. The compound of claim 19, wherein R³ is: 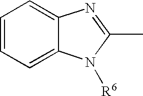
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

21. The compound of claim 20, wherein R⁶ is hydrogen or lower alkyl.

22. The compound of claim 21, wherein the compound has the structure:

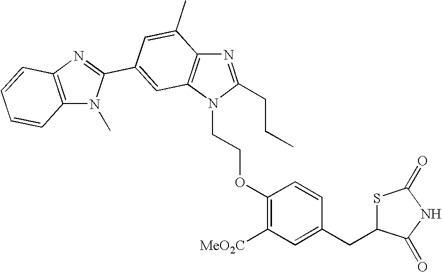

23. The compound of claim 19, wherein the compound has the structure:

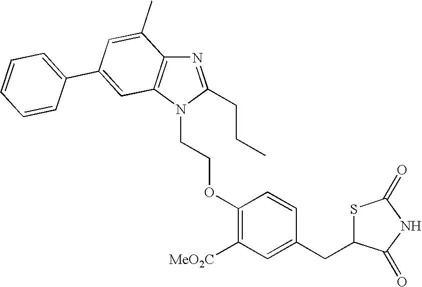

24. The compound of claim 19, wherein the compound has the structure:

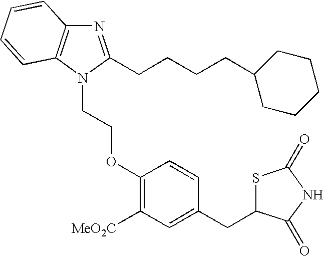

25. The compound of claim 17, wherein the compound at least partially activates PPARγ.

26. A pharmaceutical composition for the treatment or prevention of an inflammatory or metabolic disorder in a mammal comprising:
(a) a compound having the structure: 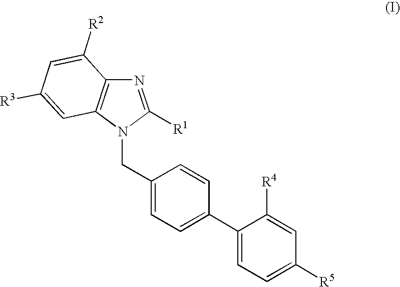
wherein R¹, R², and R³ independently are hydrogen, optionally substituted alkyl, cycloheteroalkyl, alkylheterocycloalkyl, aryl, halogen, heteroaryl, alkylheteroaryl, amino, alkylthio, alkylsulfinyl, alkylsulfonyl, alkylsulfonylamino, acyl, alkoxycarbonyl, amido, alkoxy, thiocarboxy, carbamoyl, arylalkyl, heteroalkyl or heteroarylalkyl; and
R⁴ and R⁵ independently are hydrogen, cyanate, 2-tetrazolyl, carboxyl, alkoxycarbonyl, carbamoyl, sulfonamido, or alkylsulfonamido; and
(b) a pharmaceutically acceptable vehicle.

27. The pharmaceutical composition of claim 26, wherein:
R¹ is hydrogen, halogen, methoxy, hydroxyl, methyl, ethyl, or NH₂;
R² is lower alkyl;
R³ is phenyl, a fused 2-membered heterocyclic radical or 5- or 6-membered heterocyclic ring;
R⁴ is hydrogen or cyanate; and
R⁵ is a negatively charged group.

28. The pharmaceutical composition of claim 27, wherein
R¹ is hydrogen or methyl;
R² is lower alkyl;
R³ is benzimidazole;
R⁴ is a hydrogen; and
R⁵ is alkoxycarbonyl or carboxyl.

29. The pharmaceutical composition of claim 28, wherein R³ is: 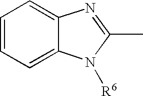
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

30. The pharmaceutical composition of claim 26, wherein:
R¹ is alkyl of 3 or more carbons;
R² is hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
R³ is aryl, 5- or 6-membered heterocyclic ring, or a fused 2-, 3-, or 4-membered heterocyclic radical;
R⁴ is a negatively charged group; and
R⁵ is a negatively charged group.

31. The pharmaceutical composition of claim 30, wherein
R¹ is propyl or butyl;
R² is lower alkyl;
R³ is phenyl, a fused 2-membered heterocyclic radical or 5- or 6-membered heterocyclic ring;
R⁴ is cyanate, 2-tetrazolyl, carboxyl, alkoxycarbonyl, carbamoyl, sulfonamido, or alkylsulfonamido; and
R⁵ is cyanate, 2-tetrazolyl, carboxyl, alkoxycarbonyl, carbamoyl, sulfonamido, or alkylsulfonamido.

32. The pharmaceutical composition of claim 31, wherein
R¹ is propyl or butyl;
R² is lower alkyl;
R³ is benzimidazole;
R⁴ is lower alkoxycarbonyl or carboxyl; and
R⁵ is lower alkoxycarbonyl or carboxyl.

33. The pharmaceutical composition of claim 32, wherein R³ is: 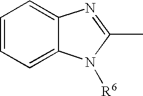
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

34. The pharmaceutical composition of claim 33, wherein R⁶ is hydrogen or lower alkyl.

35. The pharmaceutical composition of claim 34, wherein the compound has the structure:

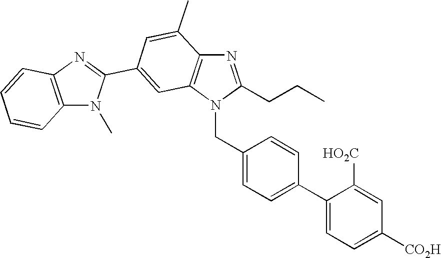

36. The pharmaceutical composition of claim 34, wherein the compound has the structure:

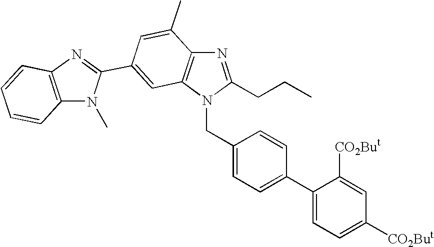

37. The pharmaceutical composition of claim 26, wherein R⁴ and R⁵ independently are hydrogen or 2-tetrazolyl.

38. The pharmaceutical composition of claim 37, wherein the compound has the structure:

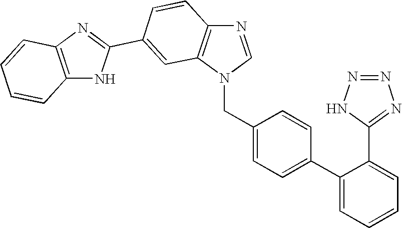

39. The pharmaceutical composition of claim 26, wherein the compound at least partially activates PPARγ.

40. The pharmaceutical composition of claim 39, wherein the compound does not inhibit activity of the AT1 receptor.

41. The pharmaceutical composition of claim 39, wherein the compound at least partically inhibits activity of the AT1 receptor.

42. The pharmaceutical composition of claim 26, wherein the inflammatory or metabolic disorder is selected from the group consisting of type 2 diabetes, metabolic syndrome, and weight gain.

43. The pharmaceutical composition of claim 26, wherein the pharmaceutical composition is formulated for oral administration.

44. The pharmaceutical composition of claim 26, wherein the pharmaceutical composition is formulated for topical administration

45. A pharmaceutical composition comprising:
(a) a compound having the structure: 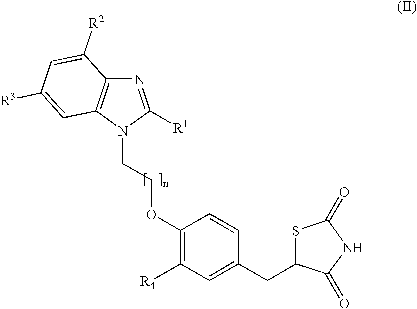
wherein R¹, R², and R³ independently are hydrogen, optionally substituted alkyl, cycloheteroalkyl, alkylheterocycloalkyl, aryl, halogen, heteroaryl, alkylheteroaryl, amino, alkylthio, alkylsulfinyl, alkylsulfonyl, alkylsulfonylamino, acyl, alkoxycarbonyl, amido, alkoxy, thiocarboxy, carbamoyl, arylalkyl, heteroalkyl or heteroarylalkyl;
n is 0 to 2; and
R⁴ is hydrogen, cyanate, 2-tetrazolyl, carboxyl, alkoxycarbonyl, amido, or sulfonamido; and
(b) a pharmaceutically acceptable vehicle.

46. The pharmaceutical composition of claim 45, wherein
R¹ is hydrogen, lower alkyl or cyclolower alkylalkyl;
R² is hydrogen, lower alkyl, halogen, hydroxyl or NH₂;
R³ is phenyl, halogen, hydrogen, amino, alkoxy, hydroxyl, 5- or 6-membered heterocyclic ring, or a fused 2-4-membered heterocyclic radical; and
R⁴ is hydrogen, cyanate, 2-tetrazolyl, carboxyl, alkoxycarbonyl, amido, or sulfonamido.

47. The pharmaceutical composition of claim 46, wherein
n is 1;
R¹ is hydrogen, methyl, ethyl or 4-cyclohexylbutyl;
R² is hydrogen, or methyl;
R³ is phenyl, halogen, or benzimidazole; and
R⁴ is loweralkoxycarbonyl or carboxyl.

48. The pharmaceutical composition of claim 47, wherein R³ is: 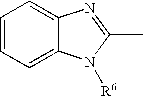
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

49. The pharmaceutical composition of claim 48, wherein R⁶ is hydrogen or lower alkyl.

50. The pharmaceutical composition of claim 49, wherein the compound has the structure:

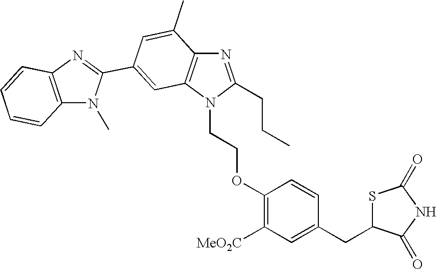

51. The pharmaceutical composition of claim 47, wherein the compound has the structure:

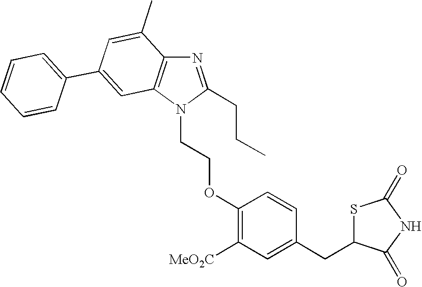

52. The pharmaceutical composition of claim 47, wherein the compound has the structure:

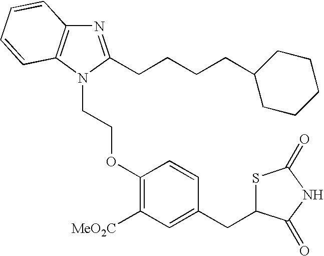

53. The pharmaceutical composition of claim 45 for the treatment or prevention of an inflammatory or metabolic disorder in a mammal.

54. The compound of claim 53, wherein the compound at least partially activates PPARγ.

55. The pharmaceutical composition of claim 54, wherein the inflammatory or metabolic disorder is selected from the group consisting of type 2 diabetes, metabolic syndrome, and weight gain.

56. The pharmaceutical composition of claim 45, wherein the pharmaceutical composition is formulated for oral administration.

57. The pharmaceutical composition of claim 45, wherein the pharmaceutical composition is formulated for topical administration

58. A method for treating or preventing an inflammatory or metabolic disorder in a mammal comprising administering to the mammal in need thereof, a therapeutically effective amount of a compound sufficient to at least partially activate a peroxisome proliferator-activated receptor (PPAR).

59. The method of claim 58, wherein the PPAR is PPARγ or a PPARγ-retinoid X receptor (PPARγ-RXR) heterodimer.

60. The method of claim 58, wherein the compound is administered orally.

61. The method of claim 58, wherein the compound is administered topically.

62. The method of claim 58, wherein the mammal is a human child, adolescent or adult.

63. The method of claim 58 wherein the therapeutically effective amount of the compound is sufficient to at least partially inhibit an activity of an angiotensin II type 1 receptor.

64. The method of claim 58, wherein the metabolic disorder is selected from the group consisting of type II diabetes, metabolic syndrome and weight gain.

65. The method of claim 58, further comprising treating at least one cardiovascular disorder associated with any component of the metabolic disorder, wherein the cardiovascular disorder is selected from the group consisting of steatohepatitis, neuropathy, nephropathy, retinopathy, retinal neovascularization, choroidal neovascularization, macular degeneration, retinal detachment, glaucoma, cataract, microangiopathy, atherosclerosis, arteriosclerosis, occlusive cerebrovascular disease, ischemic heart disease, occlusive coronary artery disease, occlusive peripheral vascular disease, stroke, peripheral arteriosclerosis, cerebral arteriosclerosis, coronary arteriosclerosis, hyperinsulinemia-induced sensory disorder, obesity, heart failure, congestive heart failure, myocardial infarction, myocardial fibrosis, angina pectoris, cerebral infarction, cardiomyopathy, renal disorders, glomerular nephritis, glomerulosclerosis, nephrotic syndrome, hypertensive nephrosclerosis, terminal renal disorders, thrombotic disease, thromboembolic disease, atheroma formation, atherogenesis.

66. The method of claim 58, wherein the compound has the structure: 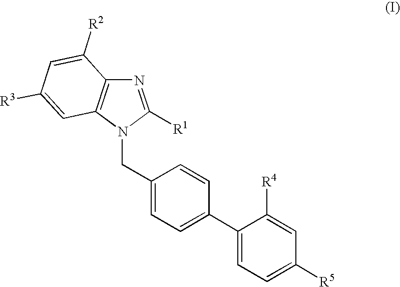
wherein R¹, R², and R³ independently are hydrogen, optionally substituted alkyl, cycloheteroalkyl, alkylheterocycloalkyl, aryl, halogen, heteroaryl, alkylheteroaryl, amino, alkylthio, alkylsulfinyl, alkylsulfonyl, alkylsulfonylamino, acyl, alkoxycarbonyl, amido, alkoxy, thiocarboxy, carbamoyl, arylalkyl, heteroalkyl or heteroarylalkyl; and
R⁴ and R⁵ independently are hydrogen, cyanate or a negatively charged group.

67. The method of claim 66, wherein:
R¹ is hydrogen, halogen, methoxy, hydroxyl, methyl, ethyl, or NH₂;
R²is lower alkyl;
R³ is phenyl, a fused 2-membered heterocyclic radical or 5- or 6-membered heterocyclic ring;
R⁴ is a hydrogen or cyanate; and
R⁵ is a negatively charged group.

68. The method of claim 67, wherein
R¹ is hydrogen or methyl;
R² is lower alkyl;
R³ is benzimidazole;
R⁴ is a hydrogen; and
R⁵ is alkoxycarbonyl or carboxyl.

69. The method of claim 68, wherein R³ is: 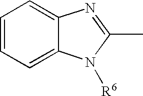
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

70. The method of claim 66, wherein the therapeutically effective amount of the compound is sufficient to at least partially inhibit an activity of an angiotensin II type 1 receptor.

71. The method of claim 70, wherein:
R¹ is alkyl of 3 or more carbons;
R² is hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
R³ is aryl, 5- or 6-membered heterocyclic ring, or a fused 2-, 3-, or 4-membered heterocyclic radical;
R⁴ is a negatively charged group; and
R⁵ is a negatively charged group.

72. The method of claim 71, wherein
R¹ is propyl or butyl;
R² is lower alkyl;
R³ is phenyl, a fused 2-membered heterocyclic radical or 5- or 6-membered heterocyclic ring;
R⁴ is cyanate, 2-tetrazolyl, carboxyl, alkoxycarbonyl, carbamoyl, sulfonamido, or alkylsulfonamido; and
R⁵ is cyanate, 2-tetrazolyl, carboxyl, alkoxycarbonyl, carbamoyl, sulfonamido, or alkylsulfonamido.

73. The method of claim 72, wherein
R¹ is propyl or butyl;
R² is lower alkyl;
R³ is benzimidazole;
R⁴ is lower alkoxycarbonyl or carboxyl; and
R⁵ is lower alkoxycarbonyl or carboxyl.

74. The method of claim 73, wherein R³ is: 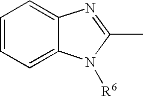
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

75. The method of claim 74, wherein R⁶ is hydrogen or lower alkyl.

76. The method of claim 75, wherein the compound has the structure:

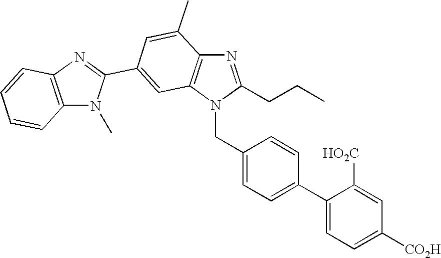

77. The method of claim 75, wherein the compound has the structure:

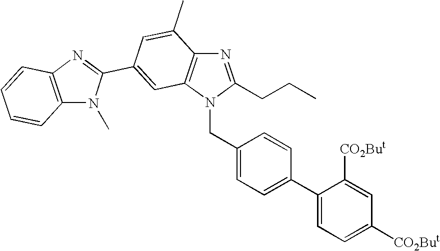

78. The method of claim 66, wherein R⁴ and R⁵ independently are hydrogen or 2-tetrazolyl.

79. The method of claim 78, wherein the compound has the structure:

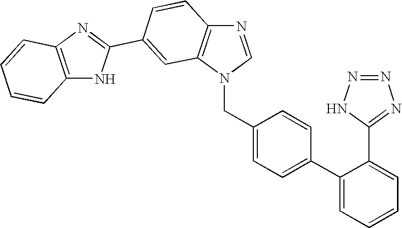

80. The method of claim 58, wherein the compound has the structure: 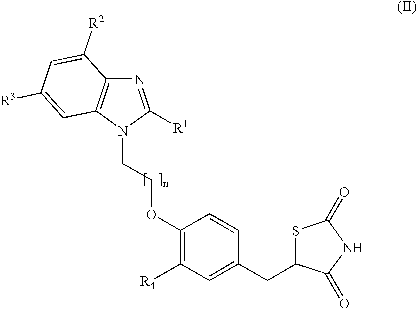
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
n is 0 to 2; and
R⁴ is hydrogen, cyanate, 2-tetrazolyl, carboxyl, alkoxycarbonyl, amido, or sulfonamido.

81. The method of claim 80, wherein
R¹ is hydrogen, lower alkyl or cyclolower alkylalkyl;
R² is hydrogen, lower alkyl, halogen, hydroxyl or NH₂;
R³ is phenyl, halogen, hydrogen, amino, alkoxy, hydroxyl, 5- or 6-membered heterocyclic ring, or a fused 2-4-membered heterocyclic radical; and
R⁴ is hydrogen, cyanate, 2-tetrazolyl, carboxyl, alkoxycarbonyl, amido, or sulfonamido.

82. The method of claim 81, wherein
n is 1;
R¹ is hydrogen, methyl, ethyl or 4-cyclohexylbutyl;
R² is hydrogen, or methyl;
R³ is phenyl, halogen, or benzimidazole; and
R⁴ is loweralkoxycarbonyl or carboxyl.

83. The method of claim 82, wherein R³ is: 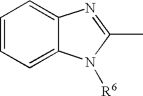
wherein R⁶ is hydrogen, optionally substituted alkyl, halogen, amino, alkylthio, alkylsulfinyl, alkylsulfonyl, alkylsulfonylamino, acyl, alkoxycarbonyl, amido, alkoxy, thiocarboxy, or carbamoyl.

84. The method of claim 83, wherein R⁶ is hydrogen or lower alkyl.

85. The method of claim 84, wherein the compound has the structure:

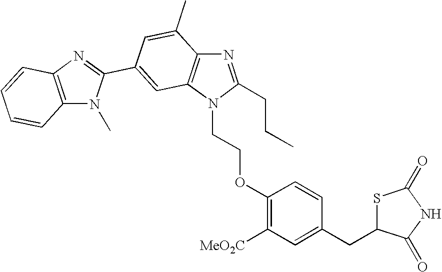

86. The method of claim 82, wherein the compound has the structure:

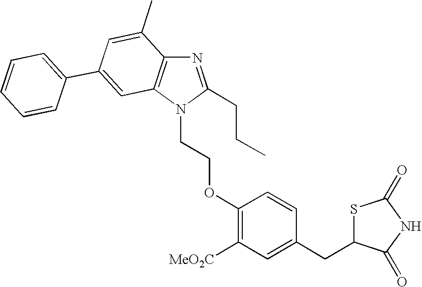

87. The method of claim 82, wherein the compound has the structure:

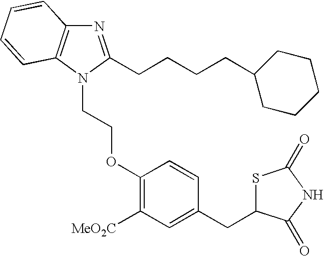

88. A method of screening a compound for capability to treat or prevent an inflammatory or metabolic disorder in a mammal, the method comprising:
(a) identifying a compound as at least partially activating a peroxisome proliferator-activated receptor (PPAR);
(b) identifying the compound as at least partially inhibiting an activity of an angiotensin II type 1 receptors; and
(c) selecting the compound as capable of treating or preventing an inflammatory or metabolic disorder.

89. The method of claim 88, wherein the PPAR is PPARγ.

90. The method of claim 88, further comprising selecting a compound that does not cause, promote, or aggravate at least one of fluid retention, peripheral edema, pulmonary edema, and congestive heart failure in the mammal.

91. The method of claim 88, wherein the compound has the structure: 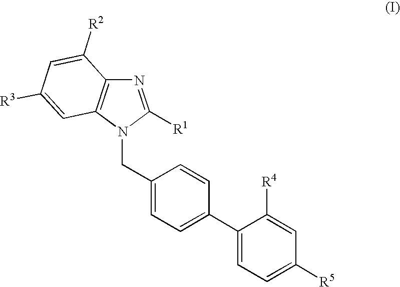
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
R⁴ and R⁵ independently are hydrogen, cyanate, or a negatively charged group.

92. The method of claim 88, wherein the compound has the structure: 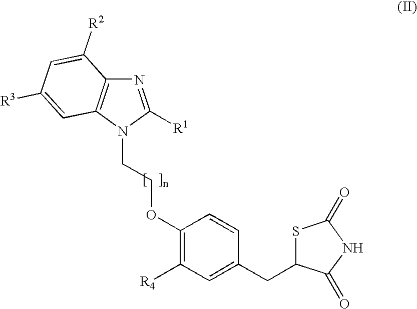
wherein R¹, R², and R³ independently are hydrogen, hydroxy, halo, amino alkylamino, dialkylamino, alkylthio, alkylsulfinyl, alkylsulfonyl, alkylsulfonylamino, acyl, alkoxycarbonyl, amido, alkoxy, thiocarboxy, carbamoyl, cyano, hydroxycarbonyl, alkylcarbonyloxy, alkylcarbonylamino, alkyl, alkenyl, alkynyl, aryl, 5- or 6-membered heterocyclic ring, or a fused 2-, 3-, or 4-membered heterocyclic radical;
n is 0 to 2; and
R⁴ is hydrogen, cyanate, 2-tetrazolyl, carboxyl, alkoxycarbonyl, amido, or sulfonamido.
